(19) United States
(12) Patent Application Publication
(10) Pub. No.: US 2025/0304544 A1
(43) Pub. Date: Oct. 2, 2025

(54) MOLEPHANTIN DERIVATIVES USEFUL IN THE TREATMENT OF CANCER

(71) Applicants: Nanyang Technological University, Singapore (SG); Nanyang Herbs Pte. Ltd., Singapore (SG)

(72) Inventors: Hoi Yeung LI, Singapore (SG); Shunsuke CHIBA, Singapore (SG); Soak Kuan LAI, Singapore (SG); Cheng Gee KOH, Singapore (SG); Toon Wah Roland ONG, Singapore (SG)

(21) Appl. No.: 18/556,616

(22) PCT Filed: Apr. 27, 2022
(86) PCT No.: PCT/SG2022/050251

§ 371 Date: Oct. 20, 2023

(30) Foreign Application Priority Data
Apr. 29, 2021 (SG) 10202104429U

(51) Int. Cl. C07D 307/93; A61K 31/365; A61P 35/00

(52) U.S. Cl. CPC C07D 307/93; A61K 31/365; A61P 35/00

(57) ABSTRACT

Disclosed herein are compounds of formula I: where R1 and R2 are as defined in the description. Also disclosed are uses of said compounds in the treatment of diseases.

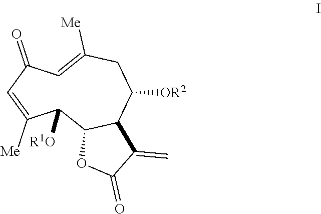

### FIELD OF INVENTION

The invention relates to molephantin derivatives, to pharmaceutical formulations comprising the molephantin derivatives, and to medical uses of the molephantin derivatives (e.g. in the treatment of cancers such as colorectal cancer and gastric cancer).

### BACKGROUND

The listing or discussion of a prior-published document in this specification should not necessarily be taken as an acknowledgement that the document is part of the state of the art or is common general knowledge.

Today, over 60% of anti-cancer drugs are derived in one way or another from plants. Notable plant-derived anti-cancer drugs that are used in clinical practice include the vina alkaloids vinblastine and vincristine, the camptothecin derivatives topotecan and irinotecan and paclitaxel (Taxol) which are isolated or derived from Catharanthus roseus G. Don. (Apocynaceae), Camptotheca acuminate Decne (Nyssaceae) and Taxus brevifolia Nutt. (Taxaceae), respectively. However, these drugs are often associated with side effects including alopecia, skin reactions, fatigue, and muscle/joint pain.

The number of new cancer cases in low- and middle-income countries is expected to rise by over 80% by 2040. Given this rise and the problems of drug resistance and adverse side effects from current therapies, there is therefore a need for new efficient anti-cancer drugs.

Elephantopus tomentosus Linn. is a species of perennial flowering plant belonging to the Asteraceae family. It is native to North America but has spread widely to the pantropical regions. In Malaysia, a decoction of the whole plant is used as a diuretic, analgesic, febrifuge, anti-helminthic and anti-inflammatory agent. The leaves of the plant are also applied externally to relieve pain.

Phytochemical studies on E. tomentosus L. have isolated compounds including triterpenes, flavonioids, alkaloids, caffeoylquinic acids and sesquiterpene lactones. Tomenphantopin-A and -B are two of the earliest sesquiterpene lactones isolated from E. tomentopus L., and demonstrated cytotoxic activity against human KB oral carcinoma cells with an ED₅₀ value of 2.5 μg/ml and 5.0 μg/ml respectively (Hayashi T, et al., Phytochemistry, vol. 26, 1987, 1065-1068). Since then, many other sesquiterpene lactones have been isolated. Tomenphantine-A and -B were found to inhibit proliferation of KB cell lines with an ED₅₀ value of 3.0 μg/ml and 2.7 μg/ml respectively (Hayashi T, et al., J Nat Prod, vol. 62, 1999, 302-304). Tomenphantopin-D and molephantin had inhibitory activities against human myeloid leukemia cell line K562 and human hepatoma cell line (SMMC-7221) with IC₅₀ values of 44.8 μM and 11.2 μM respectively for Tomenphantopin-D and 7.9 μM and 5.8 μM for molephantin, while Tomenphantopin-C, -E and -F were inactive (Mei W-L et al., Two new Germacranolides from Elephantopus tomentosus, Phytochemistry Letters, vol. 5, 2012, 800-803 and Wang B, et al., Two New Sesquiterpene Lactones from Elephantopus tomentosus, Chinese Journal of Chemistry, vol. 30, 2012, 1320-1322).

Although a number of bioactive compounds isolated from E. tomentosus L have shown cancer cell cytotoxicity and antitumor efficacy, these compounds have not yet been evaluated in clinical trials. There is also a lack of in vitro studies on the mechanistic actions of these compounds, as well as in vivo investigations in animal models. Therefore, it is not currently possible to predict whether or not any of the bioactive compounds isolated from E. tomentosus would actually have efficacy in treating cancer in vivo.

### SUMMARY OF INVENTION

The invention relates to derivatives of molephantin, which itself may be isolated from E. tomentosus L. The derivatives may be prepared by esterification and have surprisingly improved anticancer activity both in vitro and in vivo.

The invention therefore provides the following numbered clauses.
1. A compound of formula I:

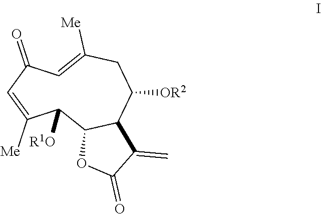

where R¹ and R² each independently represent H, —C(O)R³ or —C(O)C₁₋₆ alkyl, which latter group is unsubstituted or substituted by one or more groups selected from halo and R⁴,
   or R¹ represents —C(O)R³ or —C(O)C₁₋₆ alkyl, which latter group is unsubstituted or substituted by one or more groups selected from halo and R⁴, and R² represents —C(O)C(═CH₂)CH₃;
R³, when present, represents aryl, cycloalkyl or a heterocyclic ring system, where each of aryl, cycloalkyl and the heterocyclic ring system is unsubstituted or substituted by one or more groups selected from NO₂ and, more particularly, halo and C₁₋₃ alkyl, where C₁₋₃ alkyl is unsubstituted or substituted by one or more halo groups;
R⁴, when present, represents aryl, cycloalkyl or a heterocyclic ring system, where each of aryl, cycloalkyl and the heterocyclic ring system is unsubstituted or substituted by one or more groups selected from halo and C₁₋₃ alkyl, where C₁₋₃ alkyl is unsubstituted or substituted by one or more halo groups,
or a pharmaceutically acceptable salt or solvate thereof.
2. The compound of formula I, or a pharmaceutically acceptable salt or solvate thereof, according to Clause 1, wherein R¹ and R² each independently represent H, —C(O)R³ or —C(O)C₁₋₃ alkyl, which latter group is unsubstituted or substituted by one or more groups selected from halo and R⁴, or
   R¹ represents —C(O)R³ or —C(O)C₁₋₃ alkyl, which latter group is unsubstituted or substituted by one or more groups selected from halo and R⁴, and R² represents —C(O)C(═CH₂)CH₃, optionally wherein
   R¹ and R² each independently represent —C(O)R³ or —C(O)C₁₋₃ alkyl, which latter group is unsubstituted or substituted by one or more groups selected from halo and R⁴, or
   R¹ represents —C(O)R³ or —C(O)C₁₋₃ alkyl, which latter group is unsubstituted or substituted by one or more groups selected from halo and R⁴, and R² represents —C(O)C(═CH₂)CH₃.
3. The compound of formula I, or a pharmaceutically acceptable salt or solvate thereof, according to Clause 1 or Clause 2, wherein R³, when present, represents aryl, or a heterocyclic ring system, where each of aryl, and the heterocyclic ring system is unsubstituted or substituted by one or more groups selected from NO₂ and, more particularly, halo and C₁₋₃ alkyl, where C₁₋₃ alkyl is unsubstituted or substituted by one or more halo groups.
4. The compound of formula I, or a pharmaceutically acceptable salt or solvate thereof, according to Clause 3, wherein R³, when present, is aryl that is unsubstituted or substituted by one or more groups selected from NO₂ and, more particularly, halo, and C₁₋₃ alkyl, where C₁₋₃ alkyl is unsubstituted or substituted by one or more halo groups.
5. The compound of formula I, or a pharmaceutically acceptable salt or solvate thereof, according to Clause 4, wherein R³, when present, is phenyl that is unsubstituted or substituted by one or more groups selected from NO₂ and, more particularly, F, and C₁ alkyl, where C₁ alkyl is unsubstituted or substituted by one or more halo groups.
6. The compound of formula I, or a pharmaceutically acceptable salt or solvate thereof, according to any one of the preceding clauses, wherein R² represents —C(O)C(═CH₂)CH₃.
7. The compound of formula I, or a pharmaceutically acceptable salt or solvate thereof, according to any one of the preceding clauses, wherein R¹ represents —C(O)R³ and R² represents —C(O)R³ or —C(O)C(═CH₂)CH₃.
8. The compound of formula I, or a pharmaceutically acceptable salt or solvate thereof, according to Clause 1, selected from the list consisting of:

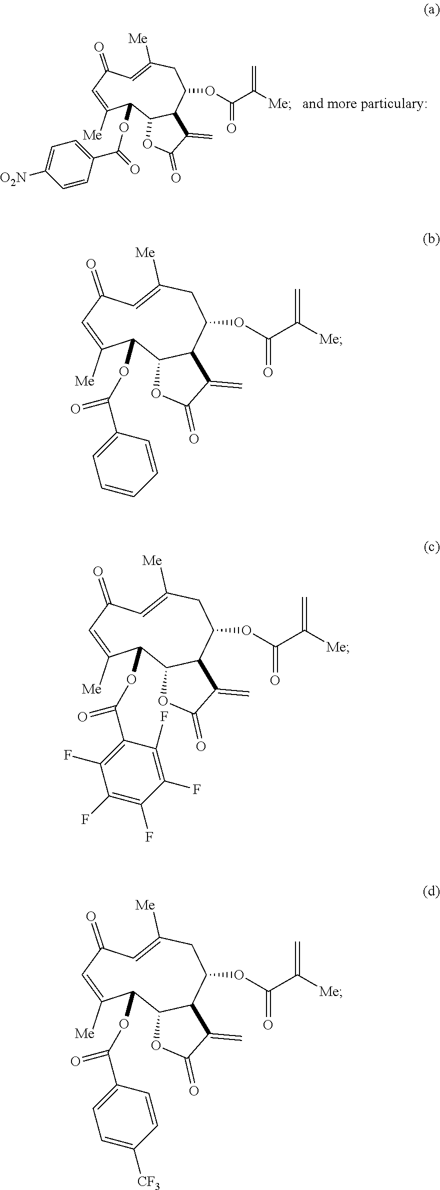

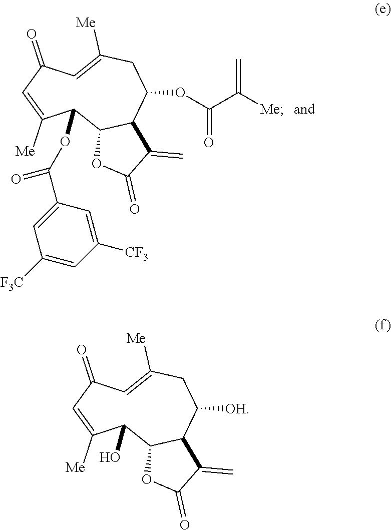

9. The compound of formula I, or a pharmaceutically acceptable salt or solvate thereof, according to Clause 8, selected from the list consisting of:

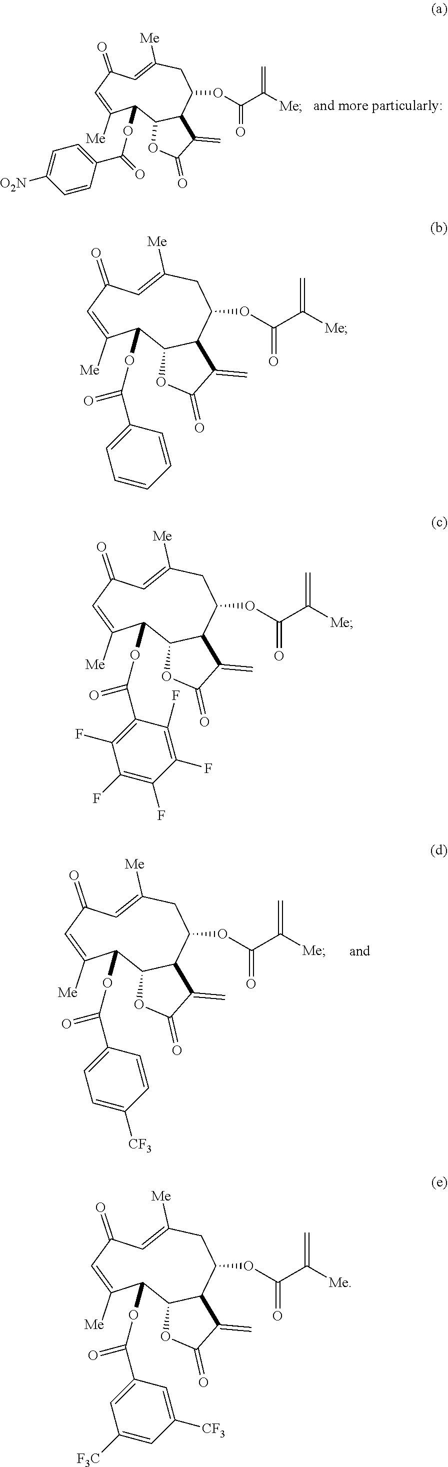

10. A pharmaceutical formulation including a compound of formula I or a pharmaceutically acceptable salt or solvate thereof, as defined in any one of Clauses 1 to 9, in admixture with a pharmaceutically-acceptable adjuvant, diluent or carrier.
11. A compound of formula I or a pharmaceutically acceptable salt or solvate thereof, as defined in any one of Clauses 1 to 9, for use in medicine.
12. Use of a compound of formula I or a pharmaceutically acceptable salt or solvate thereof, as defined in any one of Clauses 1 to 9, for the preparation of a medicament for the treatment of cancer.
13. A compound of formula I or a pharmaceutically acceptable salt or solvate thereof, as defined in any one of Clauses 1 to 9, for use in the treatment of cancer.
14. A method of treatment of cancer, which method comprises the administration of an effective amount of a compound of formula I or a pharmaceutically acceptable salt or solvate thereof, as defined in any one of Clauses 1 to 9.
15. The use according to Clause 12, the compound for use according to Clause 13, or the method according to Clause 14, wherein the cancer is selected from one or more of the group selected from adrenal cancer, anal cancer, bile duct cancer, bladder cancer, bone cancer, brain tumours, CNS tumours, breast cancer, Castleman disease, cervical cancer, colon cancer, rectum cancer, colorectal cancer, endometrial cancer, esophagus cancer, eye cancer, gallbladder cancer, gastrointestinal carcinoid tumors, gastric cancer, gastrointestinal stromal tumor (GIST), gestational trophoblastic disease, Hodgkin disease, Kaposi sarcoma, kidney cancer, laryngeal cancer, hypopharyngeal cancer, leukemia (e.g. acute lymphocytic, acute myeloid, chronic lymphocytic, chronic myeloid, chronic myelomonocytic), liver cancer, lung cancer (e.g. small cell or non-small cell), lung carcinoid tumour, lymphoma (e.g. of the skin), malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, Wilms tumour.
16. The use, compound for use or method according to Clause 15, wherein the cancer is selected from colorectal cancer and gastric cancer.

### DRAWINGS

FIGS. 1A-B show dose response curves of different cancer cell lines to generate absolute IC₅₀ value of the compounds NYH001-NYH005.

FIGS. 2A-E show colongenic assays showing colony formation of different cancer cells treated with various concentrations of compounds NYH001-NYH005. Error bars denote the standard error of the mean from three independent experiments. *P≤0.05, **P≤0.01 and ***P≤0.001.

FIG. 3 shows live cell imaging of DLD-1 cells treated with DMSO (control) or compounds NYH001-0003. Growth inhibition, mitotic arrest and cell death were triggered in cells treated with the compounds.

FIGS. 4A-D show that NYH001-NYH005 suppress the migration of cancer cells in a transwell migration assay. The figures show representative images of migrated cells (scale bar=50 μm) and quantitative analysis of migrated cells after eluting crystal violet stain and measuring absorbance at 590 nm. Data are taken from 3 independent experiments and presented as mean±SEM. *P≤0.05, **P≤0.01 and ***P≤0.001.

FIGS. 5A-B show that NYH001-NYH005 inhibit invasion of cancer cells. The figures show representative images of invaded cells (scale bar=50 μm) and quantitative analysis of invaded cells after eluting crystal violet stain and measuring absorbance at 590 nm.

FIG. 6 shows that NYH001-NYH005 induce a G2/M and S phase cell cycle arrest in DLD-1 cells. DLD-1 cells treated with DMSO or compounds for 24 hours were analysed by flow cytometry to determine cell cycle distribution.

FIG. 7 shows the dose-dependent effect of compounds NYH001-003 on the expression of apoptotic and autophagy-related proteins in DLD-1 cells. Cells were treated with DMSO and 1, 2.5 and 5 μM of either NYH001, 002 or 003 for 24 hours. Western blot was performed to check for protein levels of cleaved PARP, cleaved caspase 3 and 7, LC3B and ATG7. β-tubulin was used as a loading control.

FIG. 8 shows images of the dose dependent growth inhibition of DLD-1 tumour spheroids treated with DMSO (control) or compounds NYH001-003.

FIG. 9 shows plots of the dose dependent growth inhibition of DLD-1 tumour spheroids treated with DMSO (control) or compounds NYH001-003.

FIG. 10 shows that NYH001-NYH003 can inhibit cell motility in a dose dependent manner.

FIG. 11 shows that NYH002 treatment suppresses tumor growth in the HCT116 cells xenograft model of Example 9. Mice were treated with either vehicle control, NYH001 (25 mg/kg), NYH002 (25 mg/kg) or 5-Fu (25 mg/kg). (A) Tumor volumes throughout the course of study. Points represent the mean tumor volume in each experimental group. (B) Representative photos of tumors isolated at the experimental endpoint for each experimental group. Scale bar, 10 mm. (C) Mean tumor volume at the experimental endpoint. (D) Mean tumor weight at the experimental endpoint. (E) Mouse body weight throughout the course of study. Points represent mean body weight for mice in each treatment group All error bars indicate SEM, n=5. *P≤0.05, **P≤0.01 compared between NYH002 and vehicle control.

FIG. 12 shows that NYH002 treatment suppresses tumor growth in DLD-1 cells xenograft model of Example 9. Mice were treated with either vehicle control, NYH002 (25 mg/kg) or 5-Fu (25 mg/kg). (A) Tumor volumes throughout the course of study. Points represent the mean tumor volume in each experimental group. (B) Representative photos of tumors isolated at the experimental endpoint for each experimental group. Scale bar, 10 mm. (C) Mean tumor volume at the experimental endpoint. (D) Mean tumor weight at the experimental endpoint. (E) Mouse body weight throughout the course of study. Points represent the mean body weight for mice in each treatment group. All error bars indicate SEM, n=4. *P≤0.05, **P≤0.01 compared between NYH002 and vehicle control.

### DESCRIPTION

The invention provides a compound of formula I:

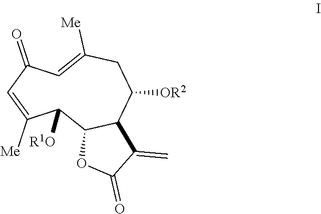

where R¹ and R² each independently represent H, —C(O)R³ or —C(O)C₁₋₆ alkyl, which latter group is unsubstituted or substituted by one or more groups selected from halo and R⁴,
   or R¹ represents —C(O)R³ or —C(O)C₁₋₆ alkyl, which latter group is unsubstituted or substituted by one or more groups selected from halo and R⁴, and R² represents —C(O)C(═CH₂)CH₃;
R³, when present, represents aryl, cycloalkyl or a heterocyclic ring system, where each of aryl, cycloalkyl and the heterocyclic ring system is unsubstituted or substituted by one or more groups selected from NO₂ and, more particularly, halo and C₁₋₃ alkyl, where C₁₋₃ alkyl is unsubstituted or substituted by one or more halo groups;
R⁴, when present, represents aryl, cycloalkyl or a heterocyclic ring system, where each of aryl, cycloalkyl and the heterocyclic ring system is unsubstituted or substituted by one or more groups selected from halo and C₁₋₃ alkyl, where C₁₋₃ alkyl is unsubstituted or substituted by one or more halo groups,
or a pharmaceutically acceptable salt or solvate thereof.

Thus, R¹ may represent H, —C(O)R³ or —C(O)C₁₋₆ alkyl, which latter group is unsubstituted or substituted by one or more groups selected from halo and R⁴, or when R² represents —C(O)C(═CH₂)CH₃, R¹ may represent —C(O)R³ or —C(O)C₁₋₆ alkyl, which latter group is unsubstituted or substituted by one or more groups selected from halo and R⁴.

In other words, when R² represents —C(O)C(═CH₂)CH₃, then R¹ is not H.

In some embodiments of the invention that may be mentioned herein, R¹ may represent —C(O)R³ or —C(O)C₁₋₆ alkyl, which latter group is unsubstituted or substituted by one or more groups selected from halo and R⁴.

In some embodiments of the invention that may be mentioned herein, R² may represent —C(O)C(═CH₂)CH₃.

In some embodiments of the invention that may be mentioned herein, R¹ and R² may each independently represent H, —C(O)R³ or —C(O)C₁₋₃ alkyl, which latter group may be unsubstituted or substituted by one or more groups selected from halo and R⁴.

In some such embodiments, R¹ and R² may each independently represent —C(O)R³ or —C(O)C₁₋₃ alkyl, which latter group may be unsubstituted or substituted by one or more groups selected from halo and R⁴.

In some embodiments of the invention that may be mentioned herein, R¹ may represent —C(O)R³ or —C(O)C₁₋₃ alkyl, which latter group may be unsubstituted or substituted by one or more groups selected from halo and R⁴, and R² may represent —C(O)C(═CH₂)CH₃.

In some such embodiments, R¹ may represent —C(O)R³ or —C(O)C₁₋₃ alkyl, which latter group may be unsubstituted or substituted by one or more groups selected from halo and R⁴, and R² may represent —C(O)C(═CH₂)CH₃.

In some embodiments of the invention that may be mentioned herein, R¹ may represent —C(O)R³ and R² may represent —C(O)R³ or —C(O)C(═CH₂)CH₃.

R³, when present, represents aryl, cycloalkyl or a heterocyclic ring system, where each of aryl, cycloalkyl and the heterocyclic ring system is unsubstituted or substituted by one or more groups selected from NO₂ and, more particularly, halo and C₁₋₃ alkyl, where C₁₋₃ alkyl is unsubstituted or substituted by one or more halo groups.

In some embodiments of the invention that may be mentioned herein, R³, when present, may represent aryl or a heterocyclic ring system, where each of aryl and the heterocyclic ring system may be unsubstituted or substituted by one or more groups selected from NO₂ and, more particularly, halo and C₁₋₃ alkyl, where the C₁₋₃ alkyl may be unsubstituted or substituted by one or more halo groups.

In some embodiments of the invention that may be mentioned herein, R³, when present, may be aryl that is unsubstituted or substituted by one or more groups selected from NO₂ and, more particularly, halo and C₁₋₃ alkyl, where C₁₋₃ alkyl is unsubstituted or substituted by one or more halo groups.

In some embodiments of the invention that may be mentioned herein, R³, when present, may be phenyl that may be unsubstituted or substituted by one or more groups selected from NO₂ and, more particularly, F and C₁ alkyl, where C₁ alkyl may be unsubstituted or substituted by one or more halo groups.

In any of the above embodiments of the invention, the substituent present on R³ may be substituted by a substituent that is not NO₂. In other words, in any of the above embodiments of the invention R³ may represent an aryl, cycloalkyl or a heterocyclic ring system (e.g. aryl or a heterocyclic ring system, such as aryl, for example phenyl), where each of aryl, cycloalkyl, the heterocyclic ring system and phenyl may be unsubstituted or substituted by one or more groups selected from halo (e.g. F) and C₁₋₃ alkyl (e.g. C₁ alkyl), where the C₁₋₃ alkyl (or C₁ alkyl) is unsubstituted or substituted by one or more halo (e.g. F) groups.

R⁴, when present, represents aryl, cycloalkyl or a heterocyclic ring system, where each of aryl, cycloalkyl and the heterocyclic ring system is unsubstituted or substituted by one or more groups selected from halo and C₁₋₃ alkyl, where C₁₋₃ alkyl is unsubstituted or substituted by one or more halo groups.

In some embodiments of the invention that may be mentioned herein, R⁴, when present, may represent aryl or a heterocyclic ring system, where each of aryl and the heterocyclic ring system may be unsubstituted or substituted by one or more groups selected from halo and C₁₋₃ alkyl, where the C₁₋₃ alkyl may be unsubstituted or substituted by one or more halo groups. In some embodiments of the invention that may be mentioned herein, R⁴, when present, may be aryl that is unsubstituted or substituted by one or more groups selected from halo and C₁₋₃ alkyl, where C₁₋₃ alkyl is unsubstituted or substituted by one or more halo groups.

In some embodiments of the invention that may be mentioned herein, R⁴, when present, may be phenyl that may be unsubstituted or substituted by one or more groups selected from F and C₁ alkyl, where C₁ alkyl may be unsubstituted or substituted by one or more halo groups.

In some embodiments of the invention where R⁴ is present, it may be present as a substituent on a methyl group. For example, the C₁₋₆ alkyl moiety and R⁴ may together represent a substituted or unsubstituted benzyl group, where the substituents are as defined above.

Where groups above are referred to as comprising “one or more” substituents, they may be substituted with one substituent or more than one substituent, for example, they may be substituted by one to six substituents, such as one to five substituents, e.g. one; two; or three substituents.

By way of example, when an alkyl group (e.g. a C₁₋₃ alkyl group such as methyl) is substituted by one or more substituents (e.g. halo groups), said alkyl group may be substituted by one, two or three substituents (e.g. halo groups). The halo groups may be fluoro groups. An example of an alkyl group substituted with one or more halo (e.g. fluoro) groups is trifluoromethyl.

As a further example, when an aryl group (e.g. a phenyl group) is substituted by one or more substituents, the aryl group may be substituted by one to five substituents (e.g. halo groups or C₁₋₃ alkyl groups, which C₁₋₃ alkyl groups may themselves be substituted or unsubstituted as defined above). The aryl group may be a phenyl group. The halo groups may be fluoro groups. The C₁₋₃ alkyl groups may be as defined above. An example of an aryl group (e.g. phenyl group) substituted with one or more halo (e.g. fluoro) groups is pentafluorophenyl.

Specific compounds according to the invention include the following:

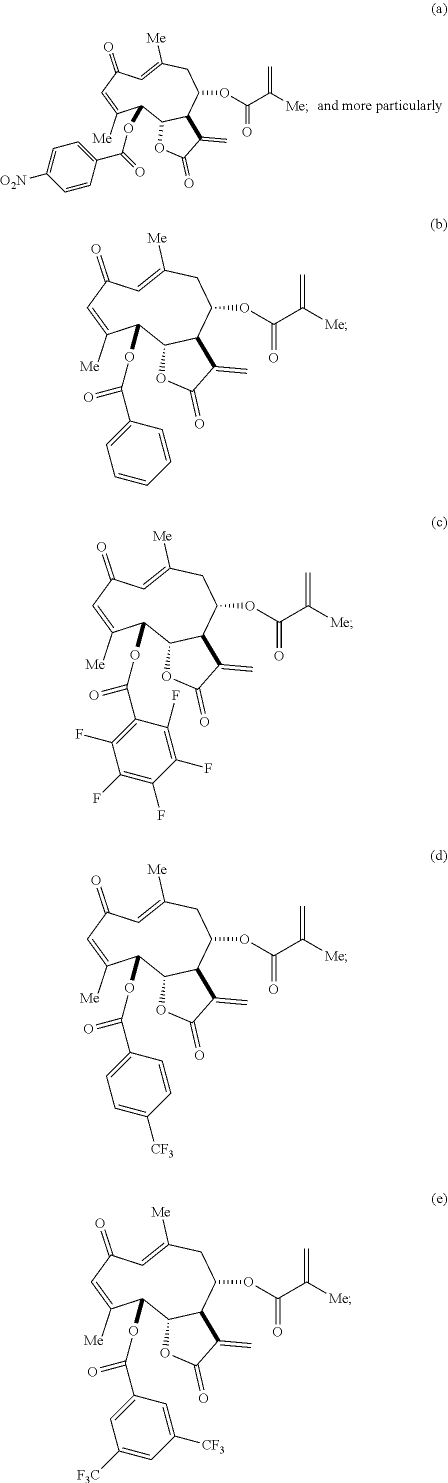

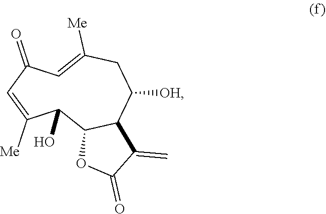

and pharmaceutically acceptable salts and solvates thereof.

In certain embodiments of the invention, the compound of formula I may be selected from:
compounds (a) to (f) above;
compounds (a) to (e) above;
compounds (b) to (f) above; and
compounds (b) to (e) above,
   and pharmaceutically acceptable salts and solvates thereof.

The invention provides a pharmaceutical formulation including a compound of formula I or a pharmaceutically acceptable salt or solvate thereof, in admixture with a pharmaceutically-acceptable adjuvant, diluent or carrier.

The compounds of formula I have anticancer activity. As such, the invention provides the following.
A compound of formula I or a pharmaceutically acceptable salt or solvate thereof, for use in medicine.
Use of a compound of formula I or a pharmaceutically acceptable salt or solvate thereof, for the preparation of a medicament for the treatment of cancer.
A compound of formula I or a pharmaceutically acceptable salt or solvate thereof, for use in the treatment of cancer.
A method of treatment of cancer, which method comprises the administration of an effective amount of a compound of formula I or a pharmaceutically acceptable salt or solvate thereof.

In each of the above use, compound for use and method of treatment, the cancer may be selected from one or more of the group selected from adrenal cancer, anal cancer, bile duct cancer, bladder cancer, bone cancer, brain tumours, CNS tumours, breast cancer, Castleman disease, cervical cancer, colon cancer, rectum cancer, colorectal cancer, endometrial cancer, esophagus cancer, eye cancer, gallbladder cancer, gastrointestinal carcinoid tumors, gastric cancer, gastrointestinal stromal tumor (GIST), gestational trophoblastic disease, Hodgkin disease, Kaposi sarcoma, kidney cancer, laryngeal cancer, hypopharyngeal cancer, leukemia (e.g. acute lymphocytic, acute myeloid, chronic lymphocytic, chronic myeloid, chronic myelomonocytic), liver cancer, lung cancer (e.g. small cell or non-small cell), lung carcinoid tumour, lymphoma (e.g. of the skin), malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, Wilms tumour.

In some embodiments of the invention that may be mentioned herein, the cancer may be selected from colorectal cancer and gastric cancer.

The word “comprising” refers herein may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise.

References herein (in any aspect or embodiment of the invention) to compounds of formula I includes references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Pharmaceutically functional derivatives” of compounds of formula I as defined herein includes ester derivatives and/or derivatives that have, or provide for, the same biological function and/or activity as any relevant compound of the invention. Thus, for the purposes of this invention, the term also includes prodrugs of compounds of formula I.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of compounds of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein a hydroxyl, amino, sulfhydryl, carboxyl or carbonyl group in a compound of formula I is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

Compounds of formula I, as well as pharmaceutically acceptable salts, solvates and pharmaceutically functional derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

The term “halo”, when used herein, includes references to fluoro, chloro, bromo and iodo.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, acyclic or cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

In particular embodiments of the invention, where the term “alkyl” is used, it may refer to an unbranched or branched, acyclic, saturated hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl).

The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pterdinyl. Particularly preferred heteroaryl groups include monocylic heteroaryl groups.

Unless otherwise specified herein, a “heterocyclic ring system” may be 4- to 14-membered, such as a 5- to 10-membered (e.g. 6- to 10-membered), heterocyclic group that may be aromatic, fully saturated or partially unsaturated, and which contains one or more heteroatoms selected from O, S and N, which heterocyclic group may comprise one or two rings. Examples of hetereocyclic ring systems that may be mentioned herein include, but are not limited to azetidinyl, dihydrofuranyl (e.g. 2,3-dihydrofuranyl, 2,5-dihydrofuranyl), dihydropyranyl (e.g. 3,4-dihydropyranyl, 3,6-dihydropyranyl), 4,5-dihydro-1H-maleimido, dioxanyl, dioxolanyl, furanyl, furazanyl, hexahydropyrimidinyl, hydantoinyl, imidazolyl, isothiaziolyl, isoxazolidinyl, isoxazolyl, morpholinyl, 1,2- or 1,3-oxazinanyl, oxazolidinyl, oxazolyl, piperidinyl, piperazinyl, pyranyl, pyrazinyl, pyridazinyl, pyrazolyl, pyridinyl, pyrimidinyl, pyrrolinyl (e.g. 3-pyrrolinyl), pyrrolyl, pyrrolidinyl, pyrrolidinonyl, 3-sulfolenyl, sulfolanyl, tetrahydrofuranyl, tetrahydropyranyl, tetrahydropyridinyl (e.g. 3,4,5,6-tetrahydropyridinyl), 1,2,3,4-tetrahydropyrimidinyl, 3,4,5,6-tetrahydropyrimidinyl, tetrahydrothiophenyl, tetramethylenesulfoxide, tetrazolyl, thiadiazolyl, thiazolyl, thiazolidinyl, thienyl, thiophenethyl, triazolyl and triazinanyl.

Unless otherwise specified herein, a “carbocyclic ring system” may be 4- to 14-membered, such as a 5- to 10-membered (e.g. 6- to 10-membered, such as a 6-membered or 10-membered), carbocyclic group that may be aromatic, fully saturated or partially unsaturated, which carbocyclic group may comprise one or two rings. Examples of carbocyclic ring systems that may be mentioned herein include, but are not limited to cyclobutyl, cyclopentyl, cyclohexyl, cyclooctyl, phenyl, naphthyl, decalinyl, tetralinyl, bicyclo[4.2.0]octanyl, and 2,3,3a,4,5,6,7,7a-octahydro-1H-indanyl. Particularly preferred carbocyclic groups include phenyl, cyclohexyl and naphthyl.

Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

The invention is illustrated by the below Examples, which are not to be considered limiting on the claims.

### EXAMPLES

### Preparatory Example 1: Extraction and Purification of Molephantin from E. Tomentosus L

The leaves from E. tomentosus L. were used in the extraction and purification of molephantin. Briefly, the crude extract was obtained by using ground freeze dried leaves or freshly collected leaves with both water and methanol as a solvent. Insoluble residue was removed from the crude extract by centrifugation and filtration. The solvent was removed later to produce a concentrated extract that was then purified using flash column chromatography with silica gel as set out below.

Crude water extracts were prepared by adding 50 g of freeze-dried powder to 1 L distilled water. The powder-water mixture was subjected to sonication using a Vibra-Cell, VCX130 sonicator at 75% amplitude (10 sec on/off) for 10 minutes. This was followed by centrifugation and filtration to remove the insoluble residue. Methanol (MeOH) was subsequently added to the filtered extract in a 1:1 (v/v) ratio. Solvent was removed from the extract using a vacuum concentrator to obtain a concentrated extract.

The extracted material (2.47 g) was re-suspended in MeOH. The silica gel (4 g) was added into the suspended material and the mixture was evaporated to prepare for dry loading the mixture onto flash column chromatography. The extracted material was purified by flash column chromatography (silica gel; CH₂Cl₂:MeOH=90:10-80:20). The resulted purified material (138 mg) was further purified by GPC (Model: LaboACE LC-5060; Column used: JAIGEL-2HR; Injection concentration: 13.8 mg/mL; Injection volume: 10 mL; Flow rate: 10 mL/min) to give molephantin (21.8 mg, 0.0629 mmol) as brown solid.

Molephantin may also be synthesised by the improved method below, which provides a greater yield.

A crude extract was obtained by using ground freeze dried leaves with ethyl acetate as a solvent. Briefly, 40 g of freeze-dried powder was added to 250 mL of ethyl acetate and the suspension was subjected to sonication (Fisherbrand® FB15051) for 30 minutes. The residue in the mixture was left to settle, and the green solution was decanted. The remaining residue was then suspended in another 250 mL ethyl acetate and sonicated for 30 minutes. This was repeated in total 5 times until the solution turns pale greenish yellow. The combined organic extracts were filtered over Celite® and concentrated in vacuo. The crude residue was purified by flash column chromatography on silica gel (n-hexane/ethyl acetate=4:1 to 1:1). Charcoal (200 mg) was then added to the purified product in ethyl acetate (15 mL) and the mixture was left to sit for 30 minutes before filtering over Celite®. Evaporation of solvent under reduced pressure followed by gel permeation chromatography (Model: LaboAce LC-5060; Column used: JAIGEL-2HR-40; Injection volume: 10 mL; Flow rate: 30 mL/min) gave two different fractions: a mixture of inseparable molephantinin and molephantin, and pure molephantin (120 mg, 0.289 mmol) as a white solid.

Molephantin is referred to hereinafter as NYH001.

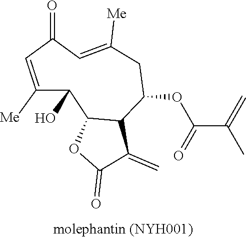

### Example 2: Synthesis of Molephantin Derivatives NYH002-NYH007

Derivatives of Molephantin were Generated by Esterification Reactions.

### Synthesis of NYH002

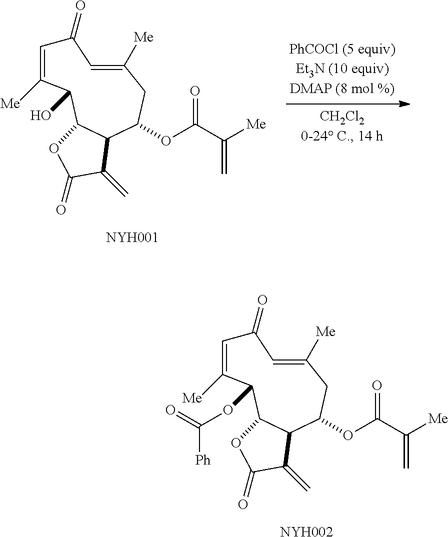

To a solution of molephantin (NYH001) (3.4 mg, 9.82 μmol, 1 equiv) in CH₂Cl₂ (1 mL) was added BzCl (6 μL, 52.07 μmol, 5 equiv), Et₃N (14 μL, 100.44 μmol, 10 equiv) and DMAP (100 μL, 1.0 mg/mL in CH₂Cl₂, 0.82 μmol, 8 mol %) at 0° C. under nitrogen, and the reaction mixture was stirred at 24° C. for 14 h. The volatile materials were then concentrated in vacuo. The resulting crude material was purified by flash column chromatography (silica gel, n-Hexane:EtOAc=70:30) to give NYH002 in 18% yield (0.8 mg, 1.8 μmol) as yellow oil, along with recovery of NYH001 in 71% yield (2.4 mg, 1.8 μmol) as brown solid.

NYH002 may also be synthesised by the improved method below, which provides a greater yield.

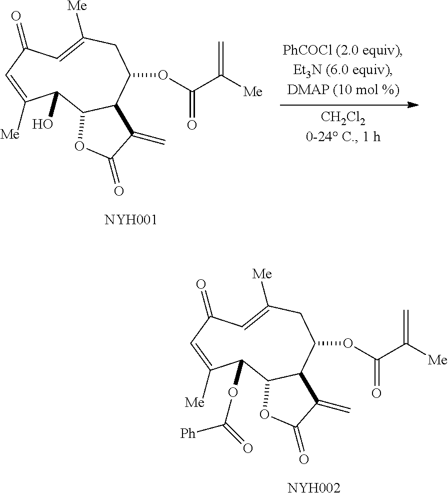

Benzoyl chloride (127 μL, 1.10 mmol, 2.0 equiv) was slowly added to the mixture of NYH001 (190.1 mg, 0.55 mmol, 1.0 equiv), DMAP (6.7 mg, 0.05 mmol, 10 mol %), triethylamine (459 μL, 3.29 mmol, 6.0 equiv) in anhydrous dichloromethane (2 mL) at 0° C. under argon. The reaction was then warmed to room temperature and left to stir for 1 hour. The mixture was then concentrated in vacuo. The crude residue was washed with saturated NaHCO₃ (10 mL) and then extracted with dichloromethane (3×10 mL). The organic layers were combined, washed with brine (10 mL), dried with MgSO₄, filtered, and carefully concentrated in vacuo. The resulting crude material was purified using flash column chromatography on silica gel (n-Hexane:EtOAc=75:25) to afford NYH002 (228 mg, 92%) as a white solid.

### Synthesis of NYH003

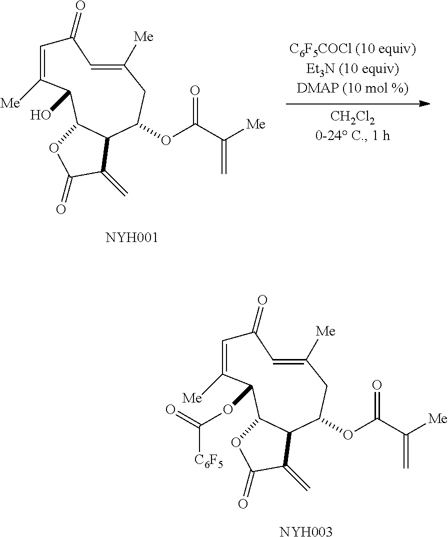

To a solution of NYH001 (4.5 mg, 12.99 μmol, 1 equiv) in CH₂Cl₂ (1 mL) was added pentafluorobenzoyl chloride (19 μL, 131.96 μmol, 10 equiv), Et₃N (18 μL, 129.14 μmol, 10 equiv) and DMAP (140 μL, 1.1 mg/mL in CH₂Cl₂, 1.26 μmol, 10 mol %) at 0° C. under nitrogen, and the reaction mixture was stirred at 24° C. for 1 h. The volatile materials were then concentrated in vacuo. The resulting crude material was purified by flash column chromatography (silica gel, n-Hexane:EtOAc=70:30) to give NYH003 in 24% yield (1.7 mg, 3.1 μmol) as colorless oil.

NYH003 may also be synthesised by the improved method below, which provides a greater yield.

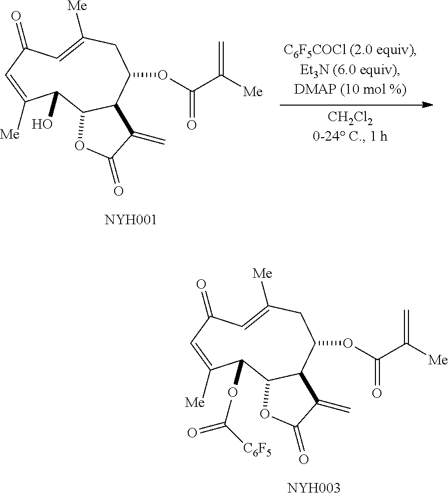

Pentafluorobenzoyl chloride (30 μL, 0.21 mmol, 2.0 equiv) was slowly added to the mixture of NYH001 (35.8 mg, 0.10 mmol, 1.0 equiv), DMAP (1.2 mg, 0.01 mmol, 10 mol %), triethylamine (86 μL, 0.62 mmol, 6.0 equiv) in anhydrous dichloromethane (1 mL) at 0° C. under argon. The reaction was then warmed to room temperature and left to stir for 1 hour. The mixture was then concentrated in vacuo. The crude residue was washed with saturated NaHCO₃ (5 mL) and then extracted with dichloromethane (3×10 mL). The organic layers were combined, washed with brine (10 mL), dried with MgSO₄, filtered, and carefully concentrated in vacuo. The resulting crude material was purified using flash column chromatography on silica gel (n-Hexane:EtOAc=75:25) to afford NYH003 (31.8 mg, 57%) as a white solid.

### Synthesis of NYH004

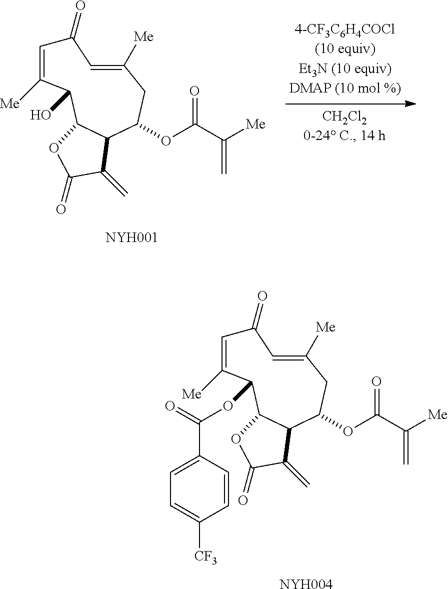

To a solution of NYH001 (7.3 mg, 21.08 μmol, 1 equiv) in CH₂Cl₂ (1 mL) was added 4-(trifluoromethyl)benzoyl chloride (31 μL, 208.69 μmol, 10 equiv), Et₃N (29 μL, 208.06 μmol, 10 equiv) and DMAP (1.3 mg, 10.64 μmol, 50 mol %) at 0° C. under nitrogen, and the reaction mixture was stirred at 24° C. for 16 h. The volatile materials were then concentrated in vacuo. The resulting crude material was purified by flash column chromatography (silica gel, n-Hexane:EtOAc=80:20) to give NYH004 in 55% yield (6.0 mg, 11.68 μmol) as light yellow solid.

NYH004 may also be synthesised by the improved method below, which provides a greater yield.

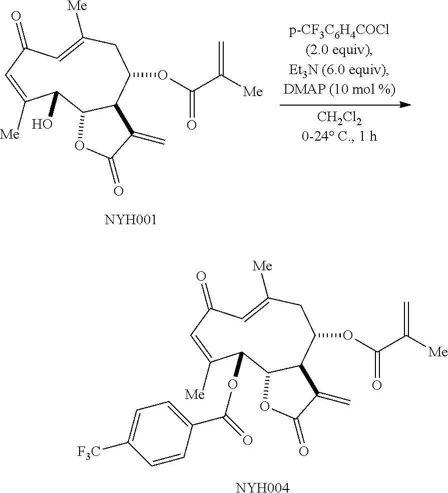

4-(Trifluoromethyl)benzoyl chloride (28 μL, 0.19 mmol, 2.0 equiv) was slowly added to the mixture of NYH001 (32.8 mg, 0.09 mmol, 1.0 equiv), DMAP (1.1 mg, 9.5 μmol, 10 mol %), triethylamine (79 μL, 0.57 mmol, 6.0 equiv) in anhydrous dichloromethane (1 mL) at 0° C. under argon. The reaction was then warmed to room temperature and left to stir for 1 hour. The mixture was then concentrated in vacuo. The crude residue was washed with saturated NaHCO₃ (5 mL) and then extracted with dichloromethane (3×10 mL). The organic layers were combined, washed with brine (10 mL), dried with MgSO₄, filtered, and carefully concentrated in vacuo. The resulting crude material was purified using flash column chromatography on silica gel (n-Hexane:EtOAc=75:25) to afford NYH004 (47.6 mg, 97%) as a white solid.

### Synthesis of NYH005

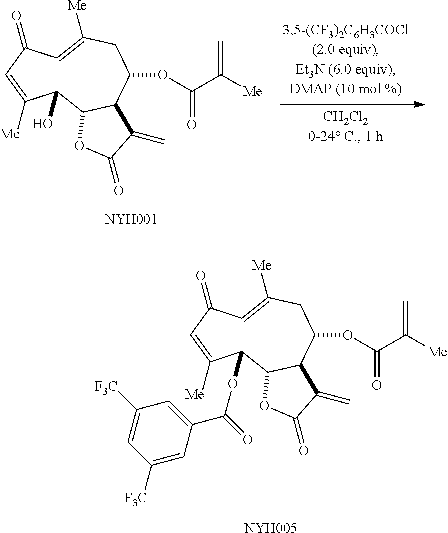

3,5-bis(trifluoromethyl)benzoyl chloride (14 μL, 0.08 mmol, 2.0 equiv) was slowly added to the mixture of NYH001 (13.8 mg, 0.04 mmol, 1.0 equiv), DMAP (0.5 mg, 4.0 μmol, 10 mol %), triethylamine (33 μL, 0.24 mmol, 6.0 equiv) in anhydrous dichloromethane (1 mL) at 0° C. under argon. The reaction was then warmed to room temperature and left to stir for 1 hour. The mixture was then concentrated in vacuo. The crude residue was washed with saturated NaHCO₃ (5 mL) and then extracted with dichloromethane (3×10 mL). The organic layers were combined, washed with brine (10 mL), dried with MgSO₄, filtered, and carefully concentrated in vacuo. The resulting crude material was purified using flash column chromatography on silica gel (n-Hexane:EtOAc=75:25) to afford NYH005 (21.1 mg, 90%) as a white solid.

### Synthesis of NYH007

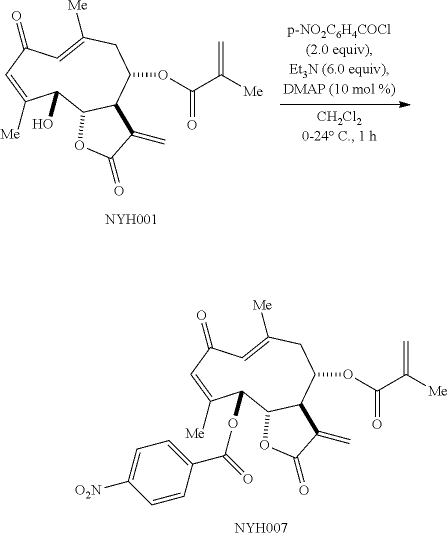

4-Nitrobenzoyl chloride (27 mg, 0.14 mmol, 2.0 equiv) was slowly added to the mixture of NYH001 (25.0 mg, 0.07 mmol, 1.0 equiv), DMAP (0.8 mg, 7.2 μL, 10 mol %), triethylamine (60 μL, 0.43 mmol, 6.0 equiv) in anhydrous dichloromethane (1 mL) at 0° C. under argon. The reaction was then warmed to room temperature and left to stir for 1 hour. The mixture was then concentrated in vacuo. The crude residue was washed with saturated NaHCO₃ (5 mL) and then extracted with dichloromethane (3×10 mL). The organic layers were combined, washed with brine (10 mL), dried with MgSO₄, filtered, and carefully concentrated in vacuo. The resulting crude material was purified using flash column chromatography on silica gel (n-Hexane:EtOAc=75:25) to afford NYH007 (31.2 mg, 87%) as a white solid.

### Example 3: Cytotoxicity Assays

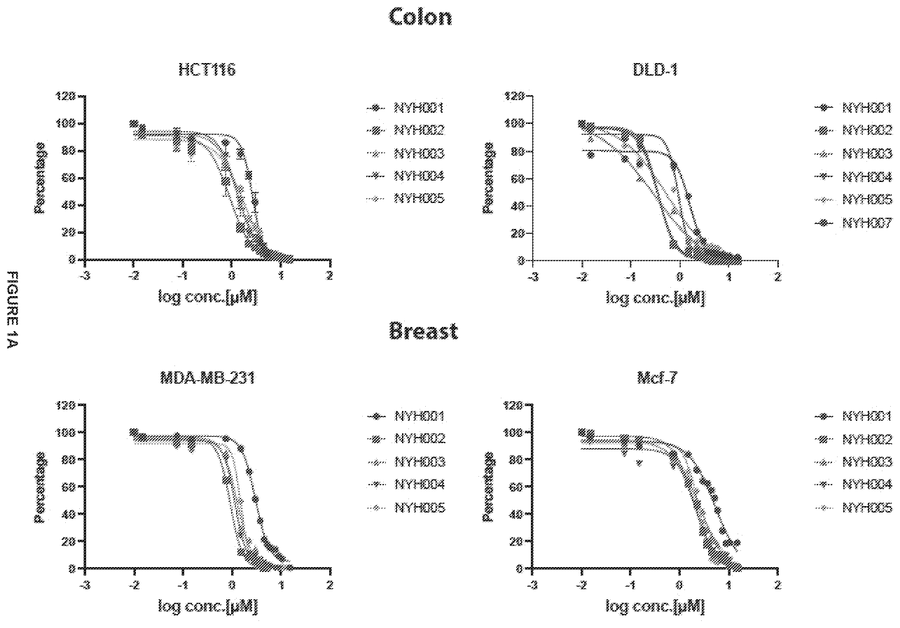

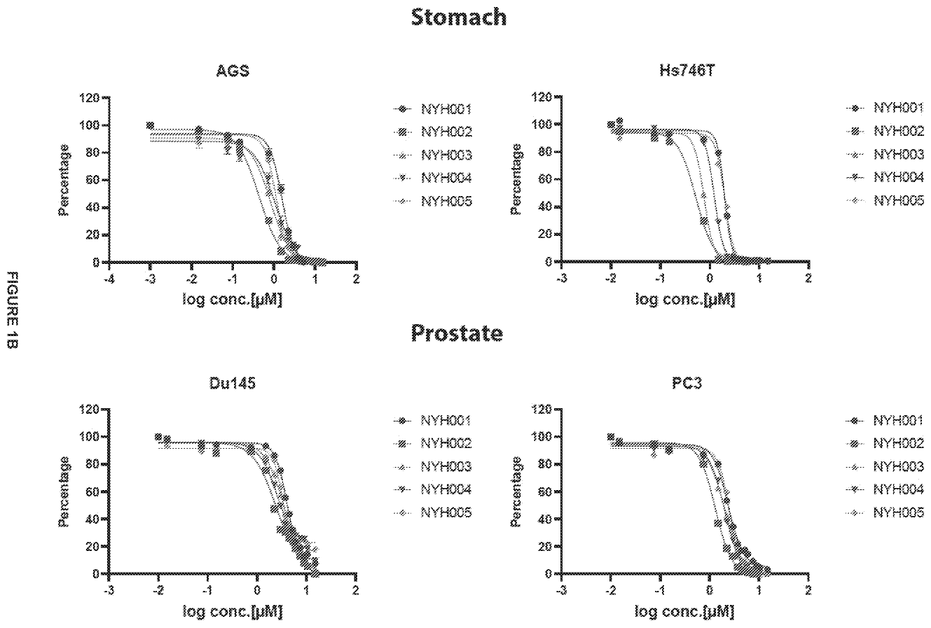

To test the cytotoxicity effect of the compounds NYH001-NYH005 in various cancer cell types, different cancer cells were treated with the compounds for 48 hours and analyzed using MTT assay. It was found that the proliferation of the cells was inhibited in a dose-dependent manner for all compounds tested. Absolute IC₅₀ values of the compounds were calculated (Table 1, FIG. 1A-C). Notably, the inhibitory effect of novel compounds NYH002-NYH005 is greater than the natural compound, NYH001 (molephantin), which indicates a higher activity in inhibiting cancer cell viability. The cytotoxic effect of NYH007 was tested in DLD-1 cells and IC₅₀ was found to be 0.35 M.

TABLE 1
IC₅₀ of NYH001-NYH005 against different cancer cell lines

| | | | IC₅₀ (μM) | | | | |
|---|---|---|---|---|---|---|---|
| Cancer | Cell lines | Histology | NYH001 | NYH002 | NYH003 | NYH004 | NYH005 |
| Colon | DLD-1 | Adenocarcinoma | 1.499 | 0.353 | 0.275 | 0.933 | 0.615 |
| | HCT116 | Carcinoma | 2.628 | 0.869 | 1.297 | 1.305 | 1.594 |
| Stomach | AGS | Adenocarcinoma | 1.544 | 0.421 | 0.782 | 1.022 | 1.117 |
| | Hs746T | Carcinoma | 1.924 | 0.468 | 0.738 | 1.193 | 1.945 |
| Breast | MDA-MB-231 | Adenocarcinoma | 3.053 | 0.909 | 1.249 | 1.138 | 1.466 |
| | Mcf-7 | Adenocarcinoma | 4.818 | 1.998 | 2.354 | 2.035 | 2.375 |
| Prostate | Du145 | Carcinoma | 4.181 | 2.362 | 3.501 | 3.002 | 3.595 |
| | PC3 | Adenocarcinoma | 2.436 | 1.275 | 1.996 | 1.973 | 2.478 |
| Lung | H1299 | Carcinoma | 2.971 | 1.353 | 1.590 | 1.666 | 1.553 |
| | A549 | Carcinoma | 15.02 | 5.08 | 5.877 | 5.226 | 5.858 |
| Liver | Huh7 | Carcinoma | 2.875 | 1.159 | 1.844 | 1.589 | 2.094 |
| | Hep3B | Carcinoma | 3.196 | 0.911 | 1.803 | 1.849 | 2.002 |

To further confirm the growth inhibitory effect of compounds on cancer cells, colony formation assays were performed. Cells were treated with various doses of the compounds and allowed to grow for 7-10 days till they form colonies. Colonies were fixed and stained with 0.5% w/v crystal violet, containing methanol. The cell culture plates were rinsed with distilled water before scanning for quantification. Colony counts were quantified using Image J software.

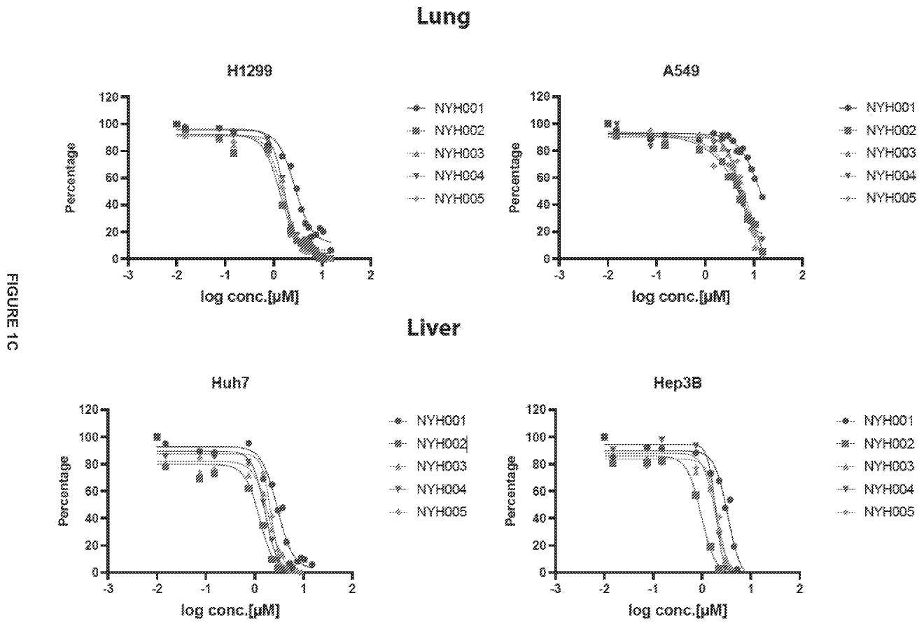

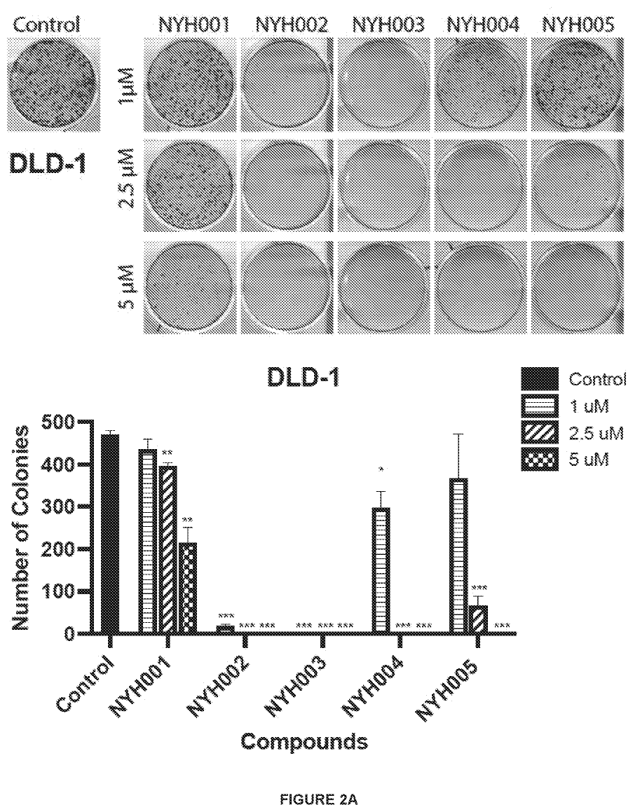

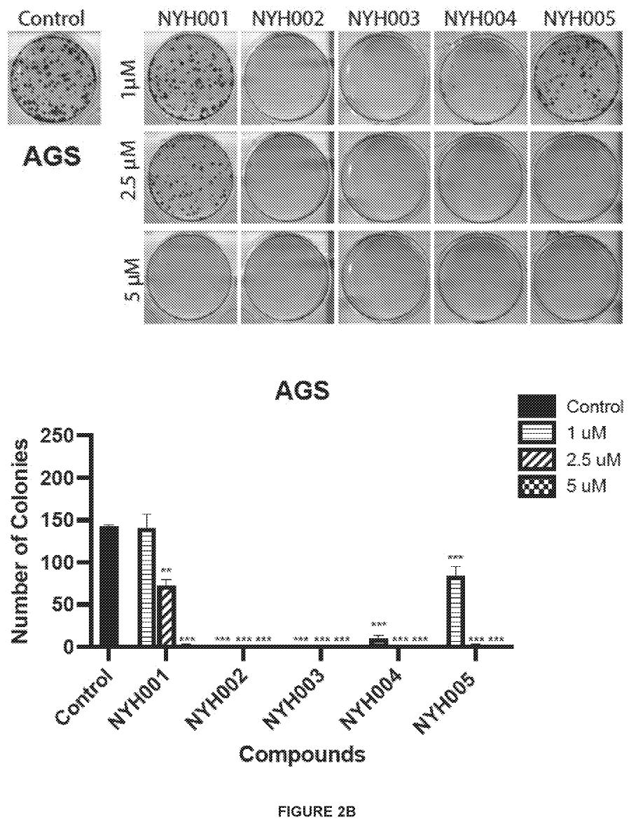

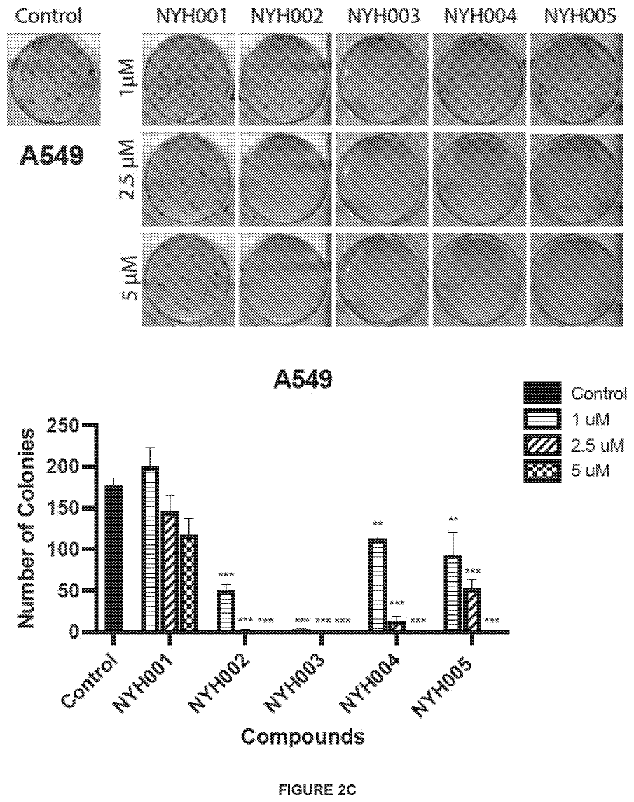

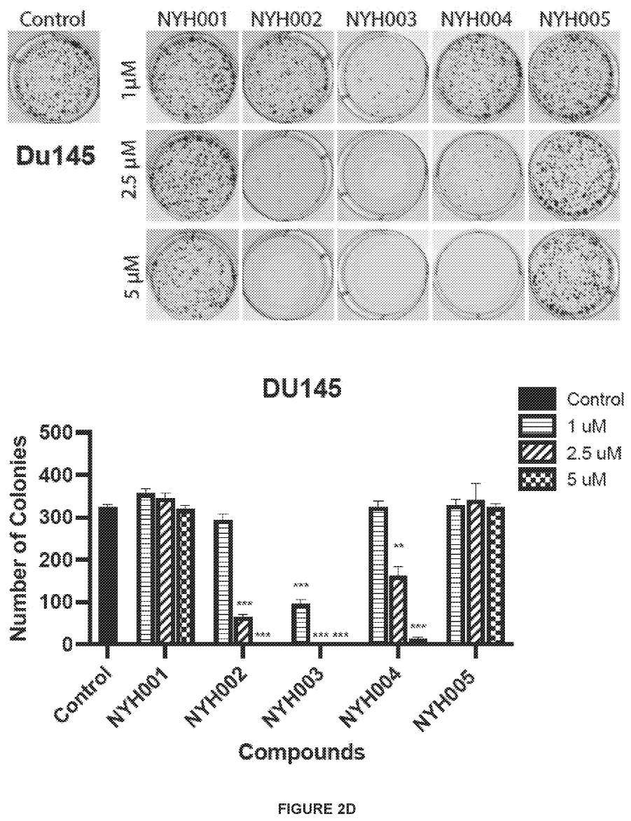

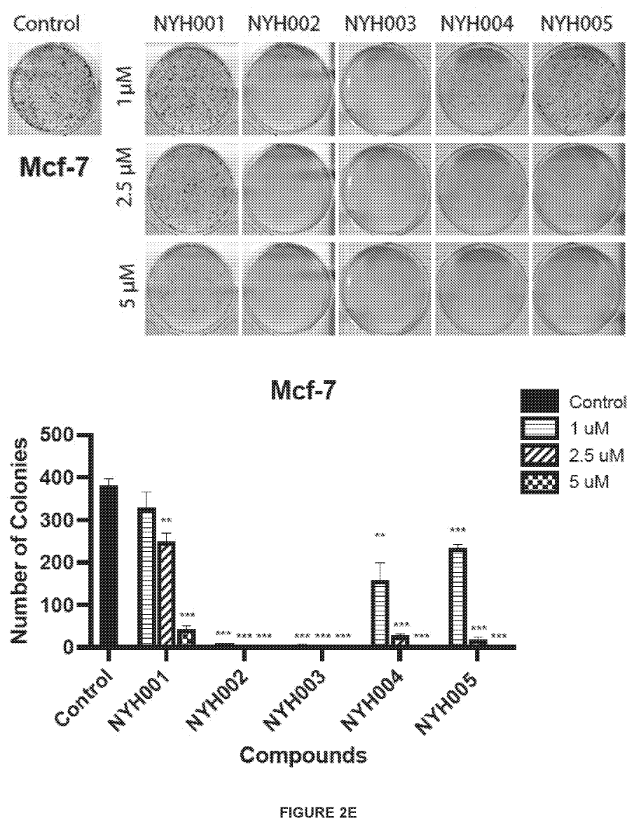

Results showed that the compounds could significantly reduce the clonogenic ability of the cells in a dose-dependent manner. Similar to the MTT cell viability assay, the novel compounds NYH002-NYH005 caused a greater effect on cell survival compared to NYH001. In particular, NYH002 and NYH003 did not result in any colonies formed for some cell lines at a concentration as low as 1 μM (FIG. 2A-E).

### Example 4: Morphological Changes by Live Cell Imaging

In order to study the cell morphological changes and cell fates, cells were seeded on a 12-well tissue culture plate, treated with compounds with different concentrations and placed on a heat-controlled stage of a Zeiss Axiovert 200M microscope. The temperature was maintained at at 37° C. and CO2 levels were maintained at 5%. Phase contrast images were acquired at 15 minutes intervals for 72 hours.

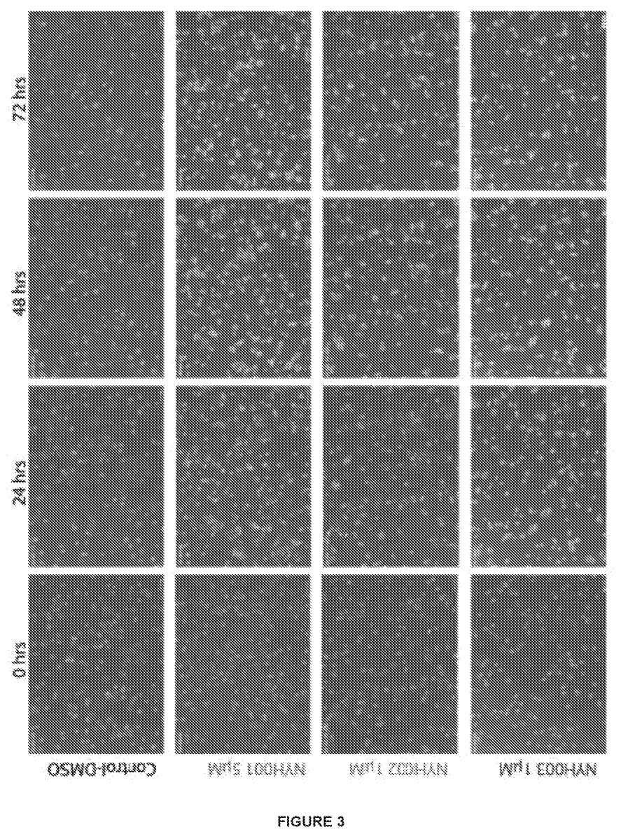

While the non-treated cells were able to grow and proliferate healthily during the course of live cell imaging, cancer cells treated with compounds NYH001-NYH003 undergo growth inhibition and cell death in a dose dependent manner. It was observed that the cancer cells exhibited cell rounding and delayed mitosis, eventually leading to cell shrinkage, membrane blebbing and cell death that resembles apoptosis (FIG. 3).

### Example 5: Cell Migration and Invasion Assays

Cell migration and invasion are key factors in driving cancer cell metastasis. To investigate the effects of the compound on cell motility and invasiveness, transwell migration and invasion assays were conducted. The transwell assays were performed using Trans-well inserts with 8 μm pore size (Corning Costar). For the migration assay, 7.5×10⁴-1.5×10⁵ cells treated with the compounds were added into the upper chamber in serum free media. In the invasion assay, the trans-well insert was first coated with Matrigel prior to cells seeding. For both assays, the lower chamber was filled with cell culture media with 10% FBS. After incubation for 24 hours at 37° C., cells that did not migrate or invade were removed from the top chamber. Cells that migrated or invaded into the bottom chamber were fixed with 4% paraformaldehyde, stained with crystal violet and imaged under a microscope. The bound crystal violet was eluted with 33% acetic acid and the eluent was transferred to a 96 well plate for measurement at absorbance 590 nm using a plate reader.

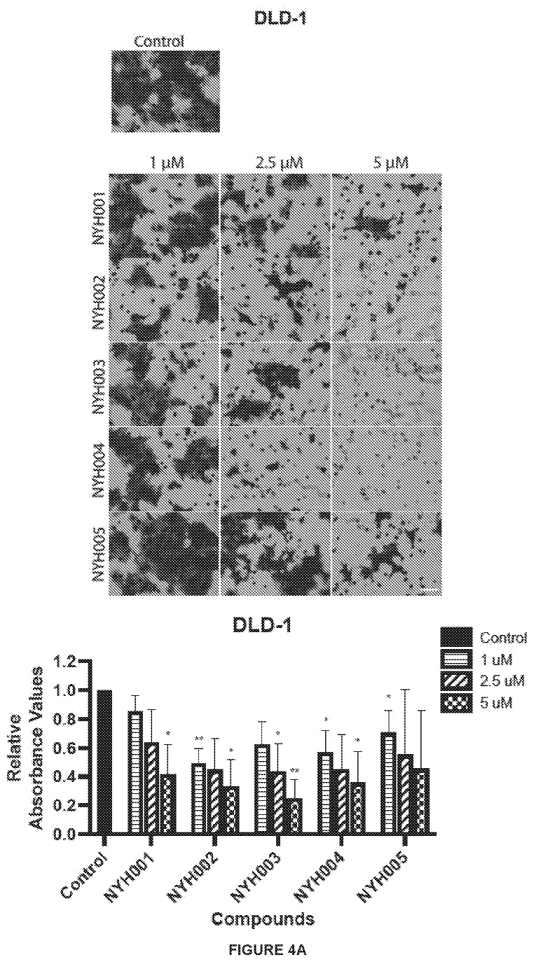

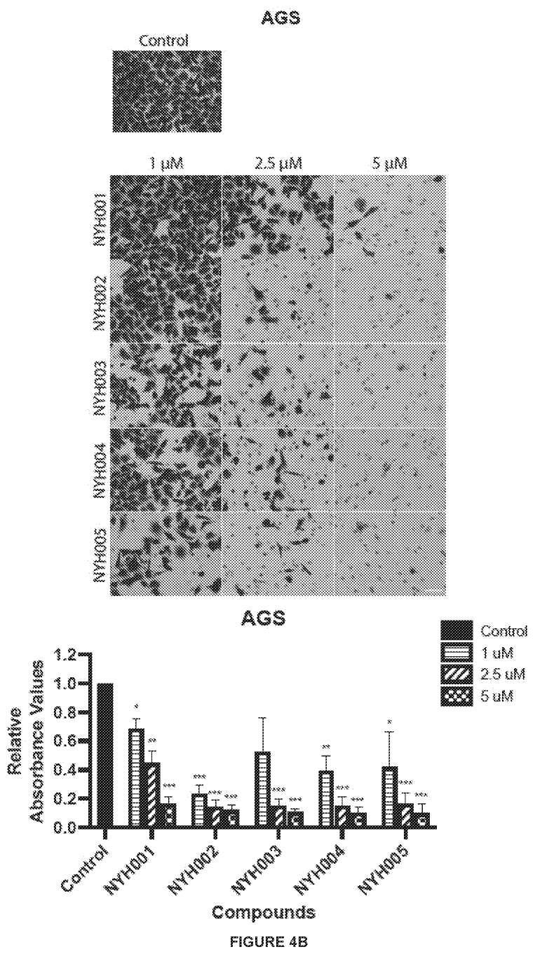

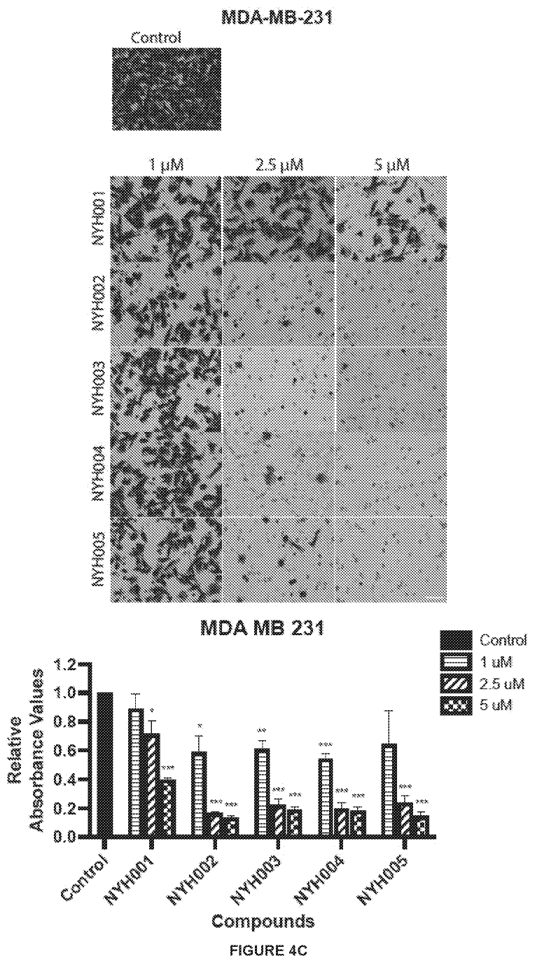

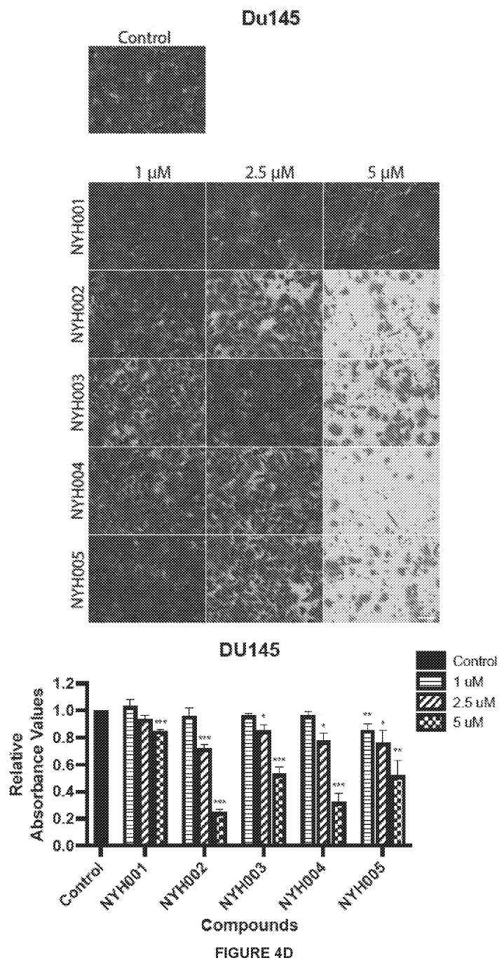

The transwell assay showed that the treatment with the single compounds inhibited the migration of the cancer cells in a dose-dependent manner (FIG. 4A-D), with NYH002-NYH005 exhibiting a stronger inhibitory effect compared to NYH001.

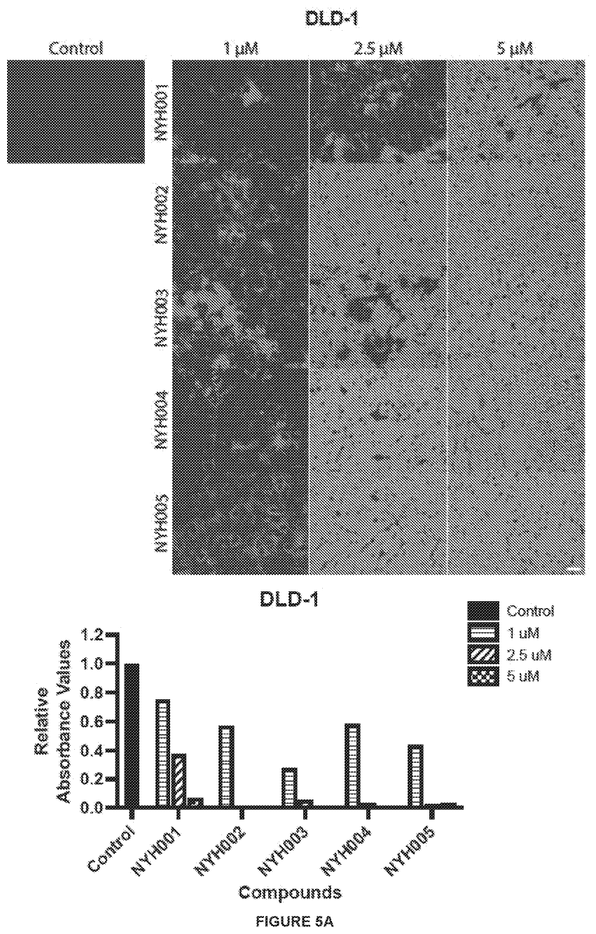

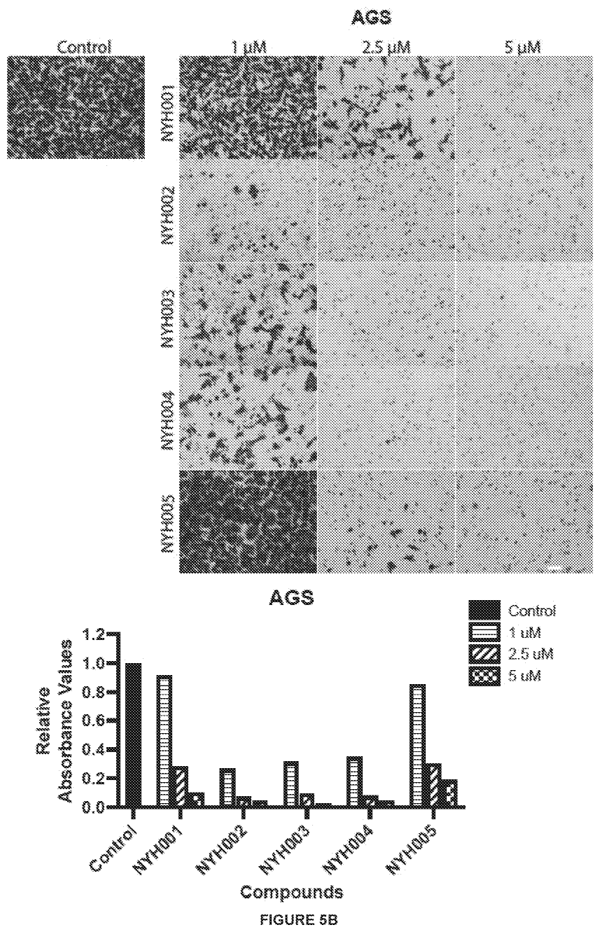

Additionally, the compounds also reduce the invasive properties of the cells in cell lines tested (FIG. 5A-B).

### Example 6: Induction of Cell Cycle Arrest and Apoptosis

The cell cycle distribution of DLD-1 cells treated with the compounds was further investigated by flow cytometry. Briefly, cells were harvested after 24 hours of treatment with NYH001-NYH005 and fixed with 70% ethanol. The cells were washed and suspended in PBS containing propidium iodide and RNase for 30 minutes. Cell cycle distribution was determined using a flow cytometer equipped with Cell Quest Pro Software. The cytometry results were analysed using FlowJo software.

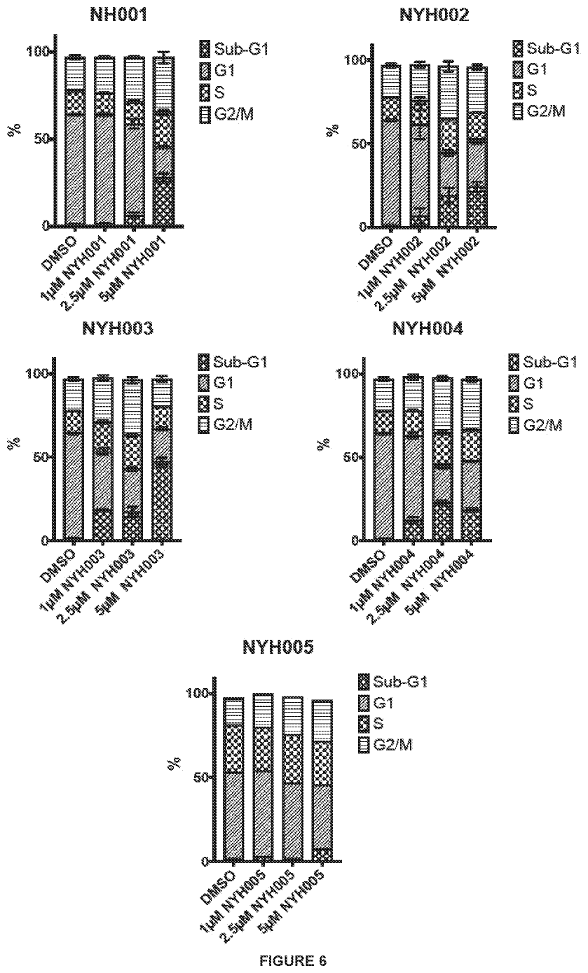

The percentage of cells in G1, S or G2/M phase after exposure to compounds was evaluated. It was found that the percentages of compound-treated DLD-1 cells in the G2/M phase were significantly higher than those in the control DMSO group, suggesting that the compounds induced a G2/M phase cell cycle arrest. There was also a slight increase in cell population in the S phase, which indicates a possibility of cell cycle delay at the S phase as well (FIG. 6).

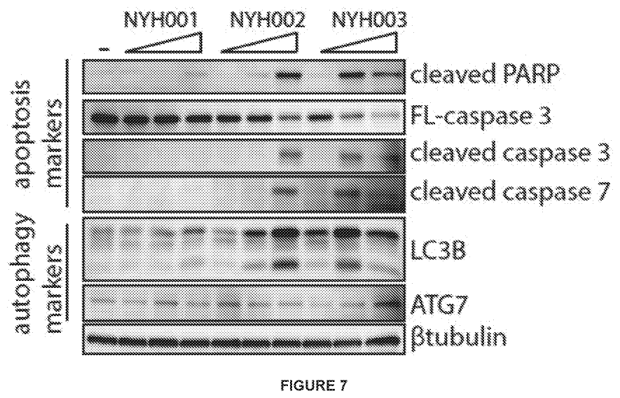

To determine the mode of cell death induced by NYH001-NYH003 in cancer cells, the expression of pro-apoptotic markers such as cleaved-PARP and cleaved caspases 3 and 7 and autophagy markers LC3B and ATG7 were quantified through western blotting. It was found that compounds NYH001-NYH003 induced the production of both the proapoptotic and autophagy markers in a dose-dependent manner in DLD-1 cells. The western blot result suggests that cell death induced by the compounds somewhat involves apoptosis and autophagy (FIG. 7).

### Example 7: Tumour Spheroid Growth Assay

A tumor spheroid growth assay was established in order to create an environment that is more physiologically relevant to tumor microenvironment and thus regarded to be more representative for in vitro drug screening. In this assay, a small tumor spheroid comprising DLD-1 cells were generated by seeding 8000 cells on a agarose coated 96-well tissue culture plate followed by centrifugation at 800 g for 5 minutes. This method allows us to generate compact spheroids with similar morphology and dimensions. The spheroids were then allowed to grow under normal conditions or treated with NYH001-003 for 15 days. 50% media replacement with or without the compounds was performed every 3 days.

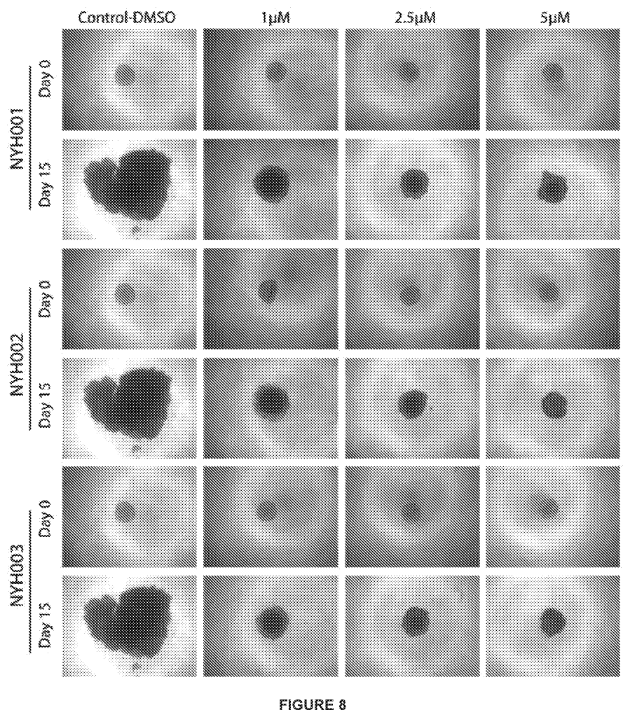

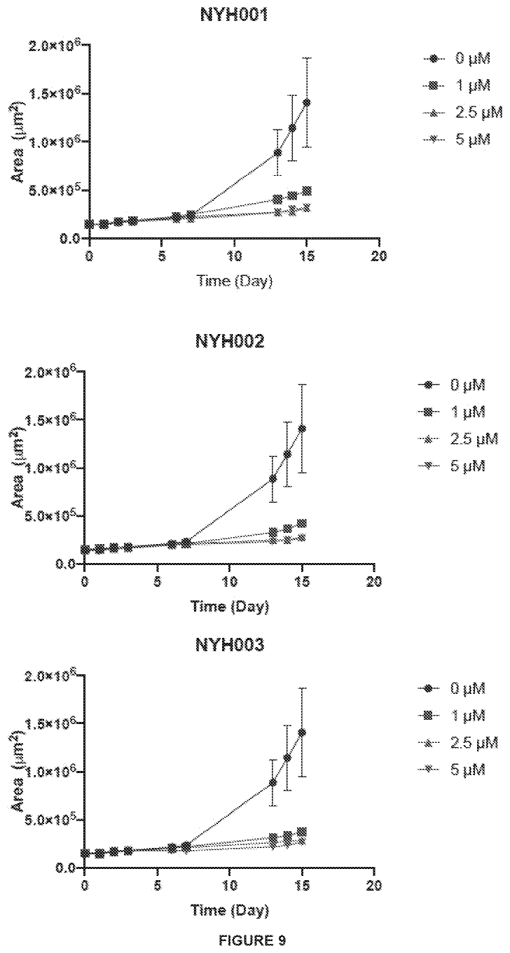

The area of the spheroid was measured using Fiji. It was evident that the growth of the spheroids treated with NYH001-NYH003 was inhibited in a dose dependent manner (FIGS. 8 and 9).

### Example 8: Wound Closure Assay

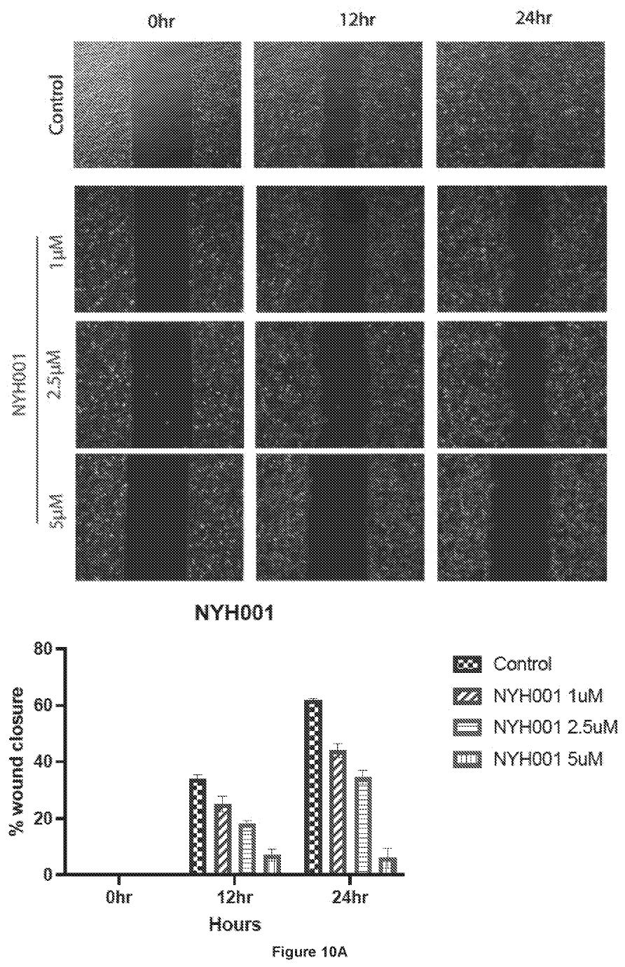

Wound closure assays were conducted to determine the effect of NYH001-003 on cell motility. AGS cells were grown in the wells of a 4-well silicon insert (Ibidi) until 100% confluency. The wells are separated by a 500 μm wall and a gap of approximated 500 μm is created after the insert is removed. Cells were washed with PBS to remove dead and floating cells prior to treatment with different concentrations of NYH001-003 or DMSO. Cell migration into the gap was monitored by live cell imaging for 24 hrs. The wound closure was analyzed by Fiji. The data showed that NYH001-003 can effectively inhibit cell motility in a dose dependent manner and hence have the potential to reduce cancer cell metastasis (FIGS. 10A-10C).

### Example 9: Inhibition of Tumour Growth in HCT116 and DLD-1 Subcutaneous Tumour Xenograft Mouse Model

Female J:Nu outbred nude mice (5-6 weeks old) were injected subcutaneously at the right flank with 1×10⁶ HCT116 or DLD-1 cells in 1:1 Hank's Balanced salt solution/Matrigel. Mice were assigned into treatment groups (4-5 mice per group) when tumour volumes reached 50-100 mm³. Vehicle control (Ethanol:Kolliphor EL:saline=1:1:8), NYH001 (25 mg/kg), NYH002 (25 mg/kg) or 5-flurouracil (5-FU, 25 mg/kg) was given once every 2 days by intraperitoneal injection for 3 weeks. Bodyweight of the mice was taken every day and tumour size was measured twice a week. Tumour sizes were calculated using the formula, volume=(length×width²)/2 mm³. At the end of the 3 weeks treatment period, the mice were sacrificed and tumours excised. All procedures were approved by the NTU Institutional Animal Care and Use Committee (IACUC) and conducted in accordance with the IACUC protocol A20001.

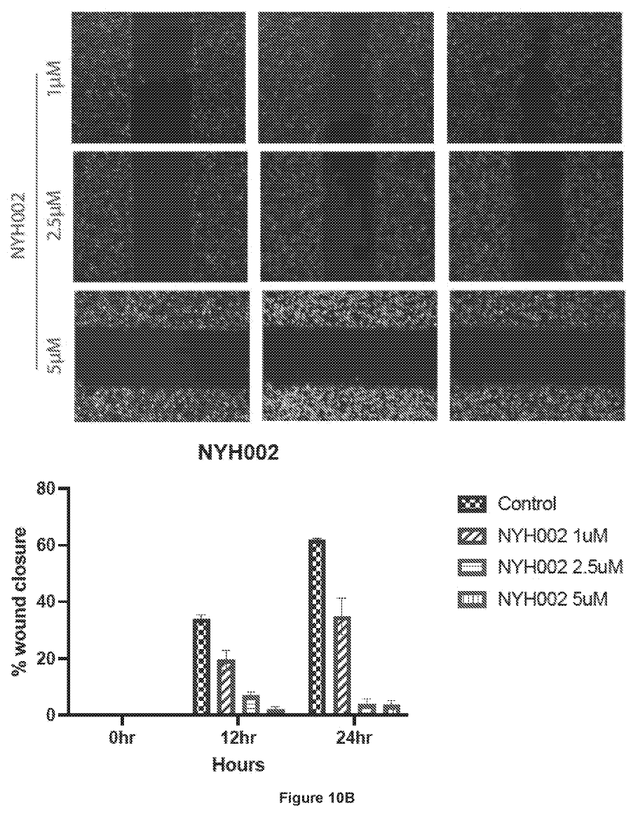

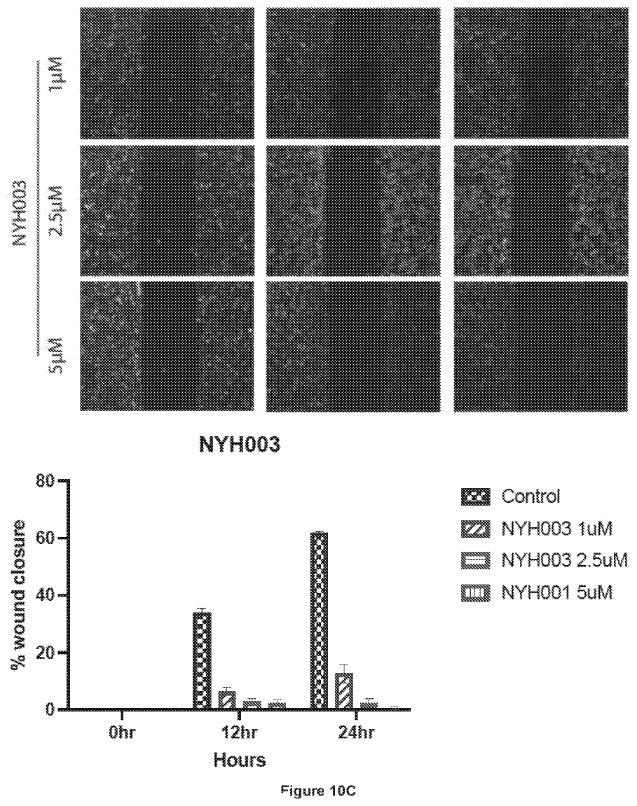

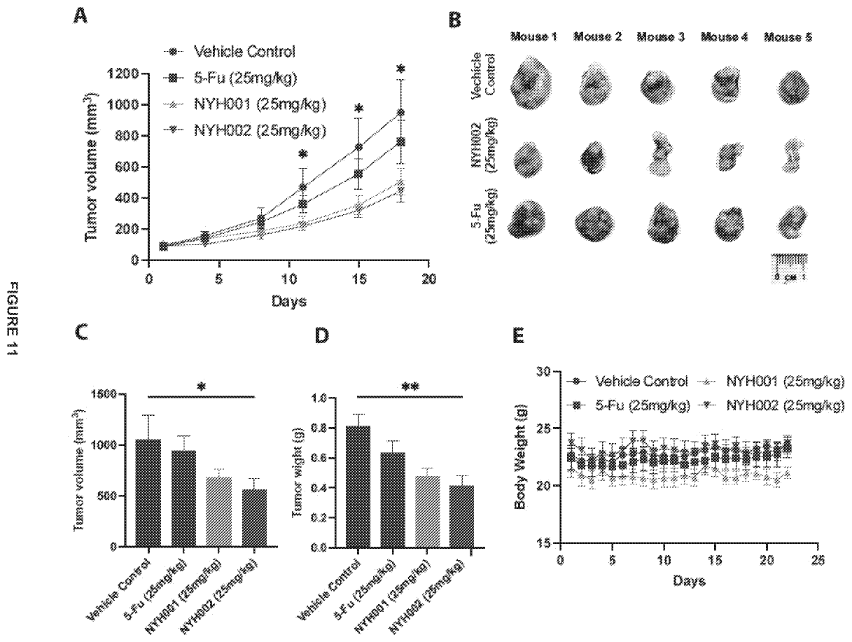

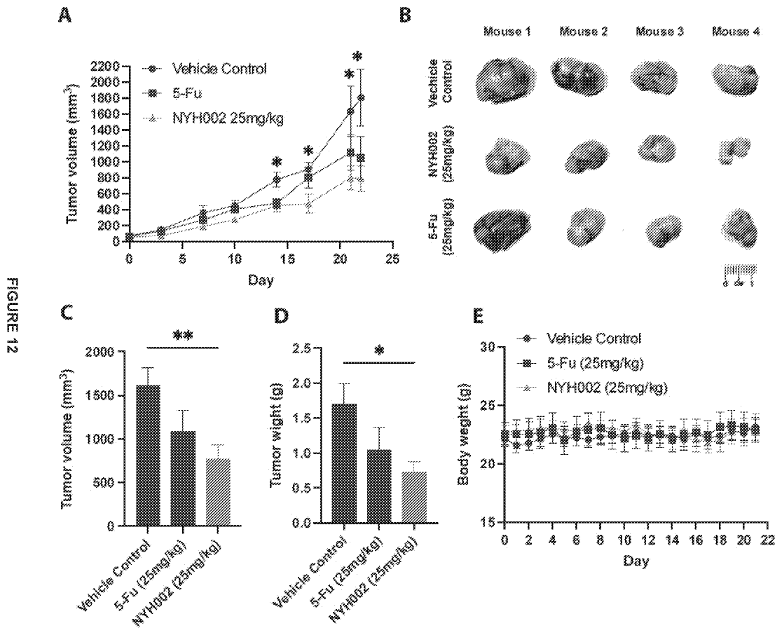

The results showed that mice treated with NYH002 treatment significantly suppress tumor growth (FIGS. 11 and 12). After 21 days of treatment, average tumor volumes were reduced by 46.5% and 51.9% for HCT116 and DLD-1 xenografted mice respectively, compared to mice that were given the vehicle control (FIGS. 11C and 12C). Average tumor weights were also reduced by 49.3% and 57.3% in HCT116 and DLD-1 xenografted mice (FIGS. 11D and 12D). The reduction in tumor sizes was notably more significant than the mice treated with 5-FU, a common chemotherapy drug used for the treatment of colon cancer. 5-FU treatment led to a reduction in tumor volume of 10.4% and 32.3% and tumor weight of 21.9% and 38.7% in HCT116 and DLD-1 xenografted mice respectively, compared to control.

The efficacy of NYH001 treatment in HCT116 xenografted mice was also evaluated. NYH001 treatment was able to inhibit tumor growth better than 5-FU treatment but at a lower efficacy compared to NYH002. Average tumor volume and weight were reduced by 35.4% and 41.7% in HCT116 xenografted mice (FIGS. 11C and 12D). All treatments did not cause adverse changes to the bodyweight throughout the course of the study (FIGS. 11E and 12E).

Overall, the results showed that NYH002 treatment has good anti-cancer efficacy in vivo with minimal toxicity and provides improved anti-cancer efficacy as compared to molephantin (NYH001) and 5-FU.

1. A compound of formula I: 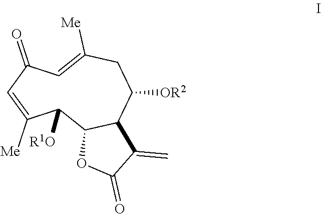
where R¹ and R² each independently represent H, —C(O)R³ or —C(O)C₁₋₆ alkyl, which latter group is unsubstituted or substituted by one or more groups selected from halo and R⁴,
or R¹ represents —C(O)R³ or —C(O)C₁₋₆ alkyl, which latter group is unsubstituted or substituted by one or more groups selected from halo and R⁴, and R² represents —C(O)C(═CH₂)CH₃;
R³, when present, represents aryl, cycloalkyl or a heterocyclic ring system, where each of aryl, cycloalkyl and the heterocyclic ring system is unsubstituted or substituted by one or more groups selected from halo, C₁₋₃ alkyl, and NO₂, where C₁₋₃ alkyl is unsubstituted or substituted by one or more halo groups;
R⁴, when present, represents aryl, cycloalkyl or a heterocyclic ring system, where each of aryl, cycloalkyl and the heterocyclic ring system is unsubstituted or substituted by one or more groups selected from halo and C₁₋₃ alkyl, where C₁₋₃ alkyl is unsubstituted or substituted by one or more halo groups,
or a pharmaceutically acceptable salt or solvate thereof.

2. The compound of formula I, or a pharmaceutically acceptable salt or solvate thereof, according to claim 1, wherein R¹ and R² each independently represent H, —C(O)R³ or —C(O)C₁₋₃ alkyl, which latter group is unsubstituted or substituted by one or more groups selected from halo and R⁴, or
R¹ represents —C(O)R³ or —C(O)C₁₋₃ alkyl, which latter group is unsubstituted or substituted by one or more groups selected from halo and R⁴, and R² represents —C(O)C(═CH₂)CH₃.

3. The compound of formula I, or a pharmaceutically acceptable salt or solvate thereof, according to claim 1, wherein R³, when present, represents aryl, or a heterocyclic ring system, where each of aryl, and the heterocyclic ring system is unsubstituted or substituted by one or more groups selected from halo, C₁₋₃ alkyl, and NO₂, where C₁₋₃ alkyl is unsubstituted or substituted by one or more halo groups.

4. The compound of formula I, or a pharmaceutically acceptable salt or solvate thereof, according to claim 3, wherein R³, when present, is aryl that is unsubstituted or substituted by one or more groups selected from halo, C₁₋₃ alkyl, and NO₂, where C₁₋₃ alkyl is unsubstituted or substituted by one or more halo groups.

5. The compound of formula I, or a pharmaceutically acceptable salt or solvate thereof, according to claim 4, wherein R³, when present, is phenyl that is unsubstituted or substituted by one or more groups selected from F, C₁ alkyl, and NO₂, where C₁ alkyl is unsubstituted or substituted by one or more halo groups.

6. The compound of formula I, or a pharmaceutically acceptable salt or solvate thereof, according to claim 1, wherein R² represents —C(O)C(═CH₂)CH₃.

7. The compound of formula I, or a pharmaceutically acceptable salt or solvate thereof, claim 1, wherein R¹ represents —C(O)R³ and R² represents —C(O)R³ or —C(O)C(═CH₂)CH₃.

8. The compound of formula I, or a pharmaceutically acceptable salt or solvate thereof, according to claim 1, selected from the list consisting of:

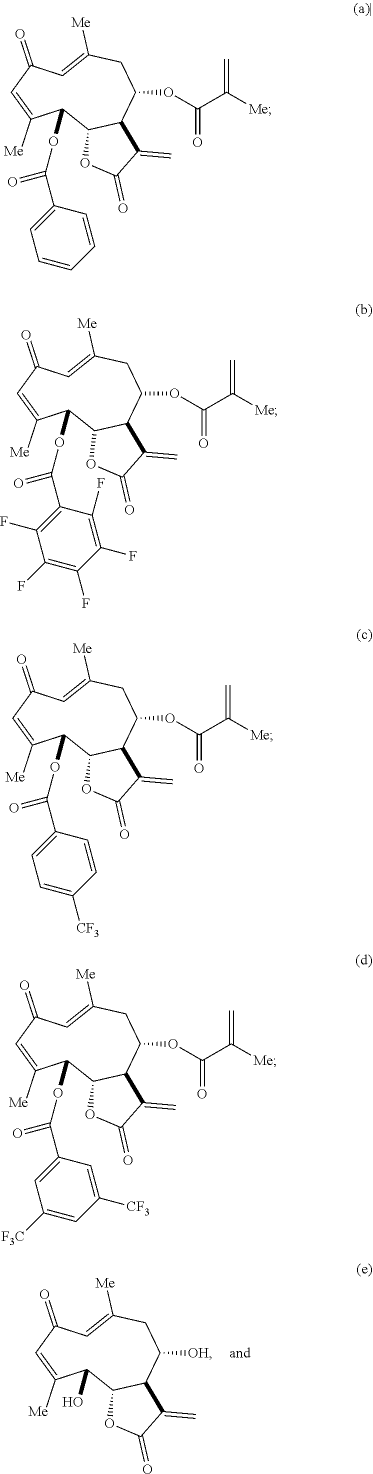

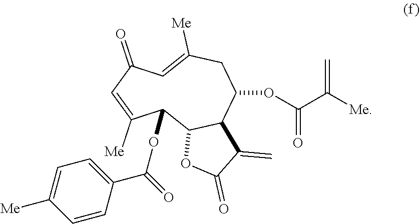

9. The compound of formula I, or a pharmaceutically acceptable salt or solvate thereof, according to claim 8, selected from the list consisting of:

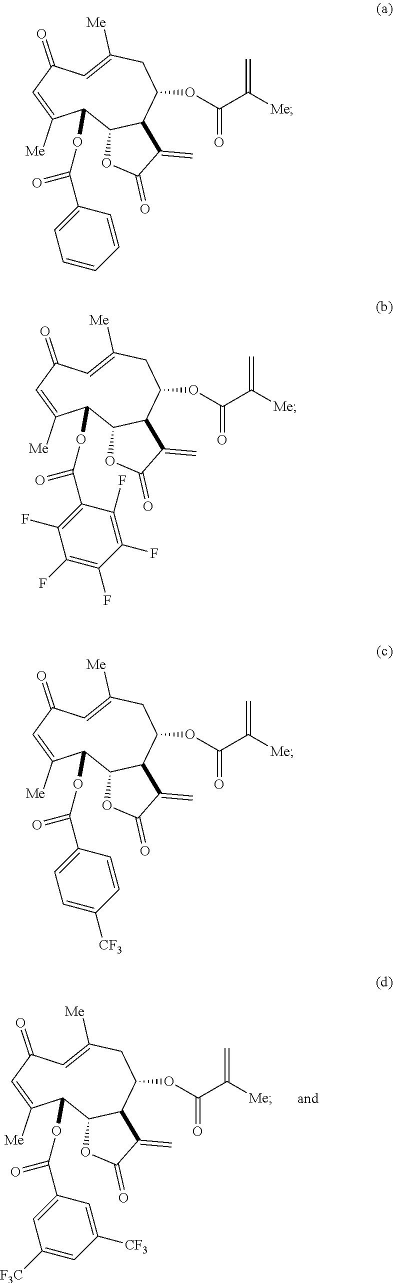

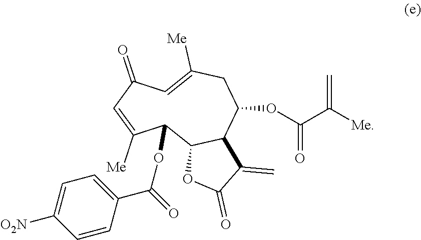

10. A pharmaceutical formulation including a compound of formula I or a pharmaceutically acceptable salt or solvate thereof, as defined in claim 1, in admixture with a pharmaceutically-acceptable adjuvant, diluent or carrier.

11. (canceled)

12. (canceled)

13. (canceled)

14. A method of treatment of cancer, which method comprises the administration of an effective amount of a compound of formula I or a pharmaceutically acceptable salt or solvate thereof, as defined in claim 1.

15. The method according to claim 14, wherein the cancer is selected from one or more of the group selected from adrenal cancer, anal cancer, bile duct cancer, bladder cancer, bone cancer, brain tumours, CNS tumours, breast cancer, Castleman disease, cervical cancer, colon cancer, rectum cancer, colorectal cancer, endometrial cancer, esophagus cancer, eye cancer, gallbladder cancer, gastrointestinal carcinoid tumors, gastric cancer, gastrointestinal stromal tumor (GIST), gestational trophoblastic disease, Hodgkin disease, Kaposi sarcoma, kidney cancer, laryngeal cancer, hypopharyngeal cancer, leukemia, liver cancer, lung cancer, lung carcinoid tumour, lymphoma, malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, Wilms tumour.

16. The method according to claim 15, wherein the cancer is selected from colorectal cancer and gastric cancer.

17. The compound of formula I, or a pharmaceutically acceptable salt or solvate thereof, according to claim 2, wherein R¹ and R² each independently represent —C(O)R³ or —C(O)C₁₋₃ alkyl, which latter group is unsubstituted or substituted by one or more groups selected from halo and R⁴.

18. The compound of formula I, or a pharmaceutically acceptable salt or solvate thereof, according to claim 2, wherein R¹ represents —C(O)R³ or —C(O)C₁₋₃ alkyl, which latter group is unsubstituted or substituted by one or more groups selected from halo and R⁴, and R² represents —C(O)C(═CH₂)CH₃.
